(19) United States
(12) Patent Application Publication
(10) Pub. No.: US 2019/0185909 A1
(43) Pub. Date: Jun. 20, 2019

(54) METHODS AND SYSTEMS FOR ANALYZING NUCLEIC ACIDS

(71) Applicant: Coyote Bioscience Co., Ltd., Beijing (CN)

(72) Inventors: Xiang LI, Beijing (CN); Chen Li, Hefei City (CN); Kun Yang, Shijiazhuang City (CN)

(21) Appl. No.: 16/053,389

(22) Filed: Aug. 2, 2018

Related U.S. Application Data
(63) Continuation of application No. PCT/CN2017/075955, filed on Mar. 8, 2017.
Application No. PCT/CN2017/075955 is a continuation-in-part of application No. PCT/CN2016/075851, filed on Mar. 8, 2016.

(51) Int. Cl. C12Q 1/6806; C12Q 1/686; C12Q 1/6886; C12Q 1/70; B01L 3/00; B01L 7/00

(52) U.S. Cl. CPC C12Q 1/6806; C12Q 1/686; C12Q 1/6886; C12Q 1/703; B01L 2300/1805; B01L 7/52; B01L 2300/0877; B01L 2300/14; B01L 3/5027

(57) ABSTRACT

The present disclosure provides methods and systems for amplifying and analyzing nucleic acid samples.

### CROSS-REFERENCE

This application is a continuation of International Application No. PCT/CN2017/075955, filed Mar. 8, 2017, which is a continuation-in-part of International Application No. PCT/CN2016/075851, filed Mar. 8, 2016, each of which are entirely incorporated herein by reference.

### BACKGROUND

Nucleic acid amplification methods may permit selected amplification and identification of nucleic acids of interest from a complex mixture, such as a biological sample. To detect a nucleic acid in a biological sample, the biological sample is typically processed to isolate nucleic acids from other components of the biological sample and other agents that may interfere with the nucleic acid and/or amplification. Following isolation of the nucleic acid of interest from the biological sample, the nucleic acid of interest can be amplified, via, for example, amplification methods such as thermal cycling based approaches (e.g., polymerase chain reaction (PCR)). Following amplification of the nucleic acid of interest, the products of amplification can be detected and the results of detection interpreted by an end-user. However, it has been tedious, time consuming and inefficient when multiple or numerous amplification reactions need to be performed.

Droplets have been proposed as containers to perform chemical and biochemical reactions (e.g., nucleic acid amplification) in confined volumes, and various methods have been developed to generate such droplets. However, these techniques often have problems associated with uneven droplet size and composition, relatively low throughput, and/or unable to generate monodisperse droplets.

### SUMMARY

Recognized herein is the need for rapid, accurate and high throughput methods and devices for analyzing nucleic acids from complex sample types. Such methods and devices may be useful, for example, in realizing fast sample-to-answer detection and management of diseases detectable via their nucleic acid.

The present disclosure provides methods and systems for efficient amplification of nucleic acids, such as RNA and DNA molecules, especially for amplifying and analyzing a large amount of different nucleic acid molecules with high throughput and/or in parallel. Amplified nucleic acid product can be detected rapidly and with high sensitivity.

In an aspect, the disclosure provides a method for analyzing a nucleic acid sample of a subject. The method comprises (a) directing (1) an aqueous fluid comprising the nucleic acid sample through a first channel and (2) a non-aqueous fluid through a second channel towards a plurality of intersections in a chip, so as to form a plurality of partitions at the plurality of intersections upon contacting between the aqueous fluid and the non-aqueous fluid. Each of the plurality of partitions includes (i) the nucleic acid sample or portion thereof, and (ii) reagents necessary for nucleic acid amplification. The method also comprises (b) subjecting the nucleic acid sample or portion thereof in each of the plurality of partitions to a nucleic acid amplification reaction under conditions that are sufficient to yield an amplification product(s) of the nucleic acid sample or portion thereof; and (c) with the plurality of partitions disposed in a collection area downstream of the plurality of intersections, simultaneously detecting signals indicative of a presence or absence of the amplification product(s) in the plurality of partitions.

In some embodiments, the method further comprises directing the plurality of partitions to the collection area. In some embodiments, the method further comprises a third channel for directing the plurality of partitions from the plurality of intersections to the collection area. In some embodiments, the third channel has a diameter that is greater than a cross-section of each of the plurality of partitions.

In some embodiments, (b) is performed in the collection area. The collection area can be included in the chip; can be substantially planar; and/or can be rotatable. In some embodiments, the collection area includes a plurality of zones, and in (c), the signals are simultaneously detected from a given zone of the plurality of zones. In some embodiments, the collection area is curvilinear (e.g., circular). In some embodiments, the collection area is tilted. In some embodiments, the collection area is removable from the chip. In some embodiments, the collection area is dimensioned to accommodate the plurality of partitions in a single layer.

In some embodiments, the plurality of partitions are droplets. In some embodiments, (b) is performed on the chip. In some embodiments, (b) comprises subjecting each of the plurality of partitions to thermal cycling. The thermal cycling can comprise cycling a temperature of each of the plurality of partitions between a first temperature and a second temperature that is greater than the first temperature. Moreover, each of the plurality of partitions can be subjected to thermal cycling using a source of thermal energy (e.g., an infrared energy source) that is external to the chip. In some embodiments, each of the plurality of partitions is subjected to thermal cycling using a source of thermal energy that is integrated with the chip. In some embodiments, a source of thermal energy is a peltier or resistive heating element. In some embodiments, a source of thermal energy is an induction heating element.

In some embodiments, the collection area comprises wells that are dimensioned to hold a single partition of the plurality of partitions. In some embodiments, each of the wells has a dimension that is less than an average diameter of a given partition of the plurality of partitions. In some embodiments, the non-aqueous fluid comprises an oil (e.g., a fluorinated oil, a mineral oil, or any oil that is useful for making droplets). In some embodiments, the non-aqueous fluid comprises a surfactant. In some embodiments, in the second channel, the non-aqueous fluid is substantially free of the sample and the reagents.

In some embodiments, the nucleic acid amplification reaction is polymerase chain reaction (PCR). In some embodiments, the nucleic acid amplification reaction is isothermal PCR. In some embodiments, the reagents include a polymerizing enzyme and primers having sequence complementary with a target nucleic acid sequence. In some embodiments, the target nucleic acid sequence is associated with a disease such as, for example, a virus or cancer. Examples of such viruses include human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, and Varicella virus. Alternatively or additionally, said target nucleic acid may be associated with food safety, prenatal testing, genetic testing, or cancer liquid biopsy, or any other application in which detection of said target nucleic acid is desirable.

In some embodiments, the partitions include detectable moieties that permit detection of the signals. In some embodiments, the detectable moieties are selected from the group consisting of TaqMan probes, TaqMan Tamara probes, TaqMan MGB probes, Lion probes, SYBR green, SYBR blue, DAPI, propidium iodine, Hoeste, SYBR gold, locked nucleic acid probes, and molecular beacons. In some embodiments, (c) comprises directing excitation energy to the plurality of partitions and detecting the signals as emissions from the plurality of partitions. In some embodiments, the signals are detected using a detector that is integrated with the chip. In some embodiments, the signals are detected using a detector that is external to the chip. In some embodiments, the detector is a charge-coupled device camera. In some embodiments, the excitation energy is provided by a source of excitation energy that is integrated with the chip. In some embodiments, the excitation energy is provided by a source of excitation energy that is external to the chip. In some embodiments, the excitation energy is provided by a light-emitting diode or a laser. In some embodiments, the signals are optical signals. In some embodiments, the signals are fluorescent signals. In some embodiments, the signals are electrostatic signals.

In some embodiments, the nucleic acid sample is from a genome of the subject. In some embodiments, the nucleic acid sample is a cell free nucleic acid sample. In some embodiments, the nucleic acid sample is cell free deoxyribonucleic acid. In some embodiments, the method further comprises providing the nucleic acid sample in the first channel without sample purification. In some embodiments, the method further comprises providing the nucleic acid sample in the first channel without ribonucleic acid (RNA) extraction. In some embodiments, the nucleic acid sample is obtained directly from the subject. In some embodiments, the nucleic acid sample is obtained directly from the subject and provided in the first channel without sample purification. In some embodiments, the nucleic acid sample is obtained directly from the subject and provided in the first channel without ribonucleic acid (RNA) extraction.

In some embodiments, the plurality of partitions is flowing at a flow rate less than about 5 ml/h through the collection area. In some embodiments, in (c), the plurality of partitions is substantially stationary. In some embodiments, the first channel includes a main channel and a plurality of secondary channels that intersect the second channel at the plurality of intersections. In some embodiments, the plurality of secondary channels are oriented at an angle from about 45° and 100° with respect to the main channel and/or the second channel. In some embodiments, the chip comprises multiple sets of the first channel, second channel, and plurality of intersections.

In some embodiments, the method further comprises, subsequent to (c), directing the plurality of partitions out of the collection area towards an outlet. In some embodiments, the outlet is under negative pressure. In some embodiments, the first channel and/or second channel are under positive pressure with respect to the outlet. In some embodiments, the aqueous fluid and non-aqueous fluid are subjected to flow using a pressure drop between the first channel and/or second channel, and the outlet that is at least about 1 psi.

In some embodiments, at the collection area, each of the plurality of partitions is at an individually addressable location. In some embodiments, the amplification product is detected at a sensitivity of at least about 90%. In some embodiments, the amplification product is detected at a specificity of at least about 90%. In some embodiments, (c) comprises simultaneously detecting signals indicative of a presence or absence of the amplification product(s) in all of the plurality of partitions.

An additional aspect of the disclosure provides a system for analyzing a nucleic acid sample of a subject. The system comprises a chip comprising a plurality of intersections of a first channel and a second channel. During use, (1) the first channel directs an aqueous fluid comprising the nucleic acid sample and (2) the second channel directs a non-aqueous fluid towards the plurality of intersections, so as to form a plurality of partitions at the plurality of intersections upon contacting between the aqueous fluid and the non-aqueous fluid. Each of the plurality of partitions includes (i) the nucleic acid sample or portion thereof, and (ii) reagents necessary for nucleic acid amplification. The system also comprises one or more computer processors that are individually or collectively programmed to (i) subject the nucleic acid sample or portion thereof in each of the plurality of partitions to a nucleic acid amplification reaction under conditions that are sufficient to yield an amplification product(s) of the nucleic acid sample or portion thereof, and (ii) with the plurality of partitions disposed in a collection area downstream of the plurality of intersections, simultaneously detect signals indicative of a presence or absence of the amplification product(s) in the plurality of partitions.

In some embodiments, the one or more computer processors are individually or collectively programmed to direct the plurality of partitions to the collection area. In some embodiments, the system further comprises a third channel for directing the plurality of partitions from the plurality of intersections to the collection area. In some embodiments, the third channel has a diameter that is greater than a cross-section of each of the plurality of partitions.

In some embodiments, the one or more computer processors are individually or collectively programmed to subject the nucleic acid sample or portion thereof in each of the plurality of partitions to the nucleic acid amplification reaction in the collection area. In some embodiments, the collection area is included in the chip; is substantially planar and/or is rotatable. In some embodiments, the collection area includes a plurality of zones. The one or more computer processors can be individually or collectively programmed to simultaneously detect the signals from a given zone of the plurality of zones. In some embodiments, the collection area is curvilinear (e.g., circular). In some embodiments, the collection area is tilted. In some embodiments, the collection area is removable from the chip. In some embodiments, the collection area is dimensioned to accommodate the plurality of partitions in a single layer.

In some embodiments, the plurality of partitions are droplets. In some embodiments, the one or more computer processors are individually or collectively programmed to subject the nucleic acid sample or portion thereof in each of the plurality of partitions to the nucleic acid amplification reaction on the chip. In some embodiments, the one or more computer processors are individually or collectively programmed to subject each of the plurality of partitions to thermal cycling to subject the nucleic acid sample or portion thereof in each of the plurality of partitions to the nucleic acid amplification reaction. Thermal cycling can comprise cycling a temperature of each of the plurality of partitions between a first temperature and a second temperature that is greater than the first temperature. In some embodiments, the one or more computer processors are individually or collectively programmed to subject each of the plurality of partitions to thermal cycling using a source of thermal energy (e.g., an infrared energy source) that is external to the chip. In some embodiments, the one or more computer processors are individually or collectively programmed to subject each of the plurality of partitions to thermal cycling using a source of thermal energy that is integrated with the chip. In some embodiments, the source of thermal energy is a Peltier or resistive heating element. In some embodiments, a source of thermal energy is an induction heating element.

In some embodiments, the collection area comprises wells that are dimensioned to hold a single partition of the plurality of partitions. In some embodiments, each of the wells has a dimension that is less than an average diameter of a given partition of the plurality of partitions. In some embodiments, the non-aqueous fluid comprises an oil (e.g., a fluorinated oil, a mineral oil, or any oil that is useful for making droplets). In some embodiments, the non-aqueous fluid comprises a surfactant. In some embodiments, in the second channel, the non-aqueous fluid is substantially free of the sample and the reagents.

In some embodiments, the nucleic acid amplification reaction is polymerase chain reaction (PCR). In some embodiments, the nucleic acid amplification reaction is isothermal PCR. In some embodiments, the reagents include a polymerizing enzyme and primers having sequence complementary with a target nucleic acid sequence. In some embodiments, the target nucleic acid sequence is associated with a disease, such as cancer or a virus. Examples of viruses include human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, and Varicella virus. Alternatively or additionally, said target nucleic acid may be associated with food safety, prenatal testing, genetic testing, or cancer liquid biopsy, or any other application in which detection of said target nucleic acid is desirable.

In some embodiments, the partitions include detectable moieties that permit detection of the signals. In some embodiments, the detectable moieties are selected from the group consisting of TaqMan probes, TaqMan Tamara probes, TaqMan MGB probes, Lion probes, SYBR green, SYBR blue, DAPI, propidium iodine, Hoeste, SYBR gold, locked nucleic acid probes, and molecular beacons. In some embodiments, the one or more computer processors are individually or collectively programmed to direct excitation energy to the plurality of partitions and detect the signals as emissions from the plurality of partitions. In some embodiments, the signals are detected using a detector that is integrated with the chip. In some embodiments, the signals are detected using a detector that is external to the chip. In some embodiments, the detector is a charge-coupled device camera. In some embodiments, the excitation energy is provided by a source of excitation energy that is integrated with the chip. In some embodiments, the excitation energy is provided by a source of excitation energy that is external to the chip. In some embodiments, the excitation energy is provided by a light-emitting diode or a laser. In some embodiments, the signals are optical signals. In some embodiments, the signals are fluorescent signals. In some embodiments, the signals are electrostatic signals.

In some embodiments, the nucleic acid sample is from a genome of the subject. In some embodiments, the nucleic acid sample is a cell free nucleic acid sample. In some embodiments, the nucleic acid sample is cell free deoxyribonucleic acid. In some embodiments, the one or more computer processors are individually or collectively programmed to provide the nucleic acid sample in the first channel without sample purification. In some embodiments, the one or more computer processors are individually or collectively programmed to provide the nucleic acid sample in the first channel without ribonucleic acid (RNA) extraction. In some embodiments, the one or more computer processors are individually or collectively programmed to simultaneously detect the signals while the plurality of partitions is flowing at a flow rate less than about 5 ml/h through the collection area. In some embodiments, the one or more computer processors are individually or collectively programmed to simultaneously detect the signals while the plurality of partitions is substantially stationary.

In some embodiments, the first channel includes a main channel and a plurality of secondary channels that intersect the second channel at the plurality of intersections. In some embodiments, the plurality of secondary channels are oriented at an angle from about 45° and 100° with respect to the main channel and/or the second channel. In some embodiments, the chip comprises multiple sets of the first channel, second channel, and plurality of intersections. In some embodiments, the one or more computer processors are individually or collectively programmed to direct the plurality of partitions out of the collection area towards an outlet.

In some embodiments, the outlet is under negative pressure. In some embodiments, the first channel and/or second channel are under positive pressure with respect to the outlet. In some embodiments, the one or more computer processors are individually or collectively programmed to subject the aqueous fluid and non-aqueous fluid to flow using a pressure drop between the first channel and/or second channel, and the outlet that is at least about 1 psi. In some embodiments, the collection area includes an individually addressable location for each of the plurality of partitions. In some embodiments, the amplification product is detected at a sensitivity of at least about 90%. In some embodiments, the amplification product is detected at a specificity of at least about 90%. In some embodiments, the one or more computer processors are individually or collectively programmed to simultaneously detect signals indicative of a presence or absence of the amplification product(s) in all of the plurality of partitions.

An additional aspect of the disclosure provides a method for analyzing a nucleic acid sample of a subject. The method comprises (a) forming a plurality of partitions upon contact between an aqueous fluid comprising the nucleic acid sample and a non-aqueous fluid. Each of the plurality of partitions includes (i) the nucleic acid sample or portion thereof, and (ii) reagents necessary for nucleic acid amplification. The method also comprises (b) subjecting the nucleic acid sample or portion thereof in each of the plurality of partitions to a nucleic acid amplification reaction under conditions that are sufficient to yield an amplification product(s) of the nucleic acid sample or portion thereof; and (c) subsequent to (b), with the plurality of partitions disposed in a collection area that is substantially planar, simultaneously detecting signals indicative of a presence or absence of the amplification product(s) in the plurality of partitions.

In some embodiments, the method further comprises directing the plurality of partitions to the collection area. In some embodiments, (b) is performed in the collection area. In some embodiments, the collection area is included in the chip. In some embodiments, the collection area includes a plurality of zones, and in (c), the signals are simultaneously detected from a given zone of the plurality of zones. In some embodiments, the collection area is dimensioned to accommodate the plurality of partitions in a single layer. In some embodiments, (b) is performed on the chip.

An additional aspect of the disclosure provides a system for analyzing a nucleic acid sample of a subject. The system comprises a chip comprising a first channel and a second channel meeting at an intersection. During use, (1) the first channel directs an aqueous fluid comprising the nucleic acid sample and (2) the second channel directs a non-aqueous fluid towards the intersection, so as to form a plurality of partitions at the intersection upon contacting between the aqueous fluid and the non-aqueous fluid. Each of the plurality of partitions includes (i) the nucleic acid sample or portion thereof, and (ii) reagents necessary for nucleic acid amplification. The system also comprises one or more computer processors that are individually or collectively programmed to (i) subject the nucleic acid sample or portion thereof in each of the plurality of partitions to a nucleic acid amplification reaction under conditions that are sufficient to yield an amplification product(s) of the nucleic acid sample or portion thereof, and (ii) subsequent to (i), with the plurality of partitions disposed in a collection area that is substantially planar, simultaneously detect signals indicative of a presence or absence of the amplification product(s) in the plurality of partitions.

An additional aspect of the disclosure provides a method for analyzing a nucleic acid sample of a subject. The method comprises (a) forming a plurality of partitions upon contact between an aqueous fluid comprising the nucleic acid sample and a non-aqueous fluid. Each of the plurality of partitions includes (i) the nucleic acid sample or portion thereof, and (ii) reagents necessary for nucleic acid amplification. The method also comprises (b) subjecting the nucleic acid sample or portion thereof in each of the plurality of partitions to a nucleic acid amplification reaction under conditions that are sufficient to yield an amplification product(s) of the nucleic acid sample or portion thereof; and (c) subsequent to (b), simultaneously detecting signals indicative of a presence or absence of the amplification product(s) in the plurality of partitions while the plurality of partitions are immobilized by wells in a collection area. Each of the wells can have a dimension that is less than an average diameter of a given partition of the plurality of partitions.

In some embodiments, the method further comprises directing the plurality of partitions to the collection area. In some embodiments, (b) is performed in the collection area. In some embodiments, the collection area is included in the chip. In some embodiments, the collection area includes a plurality of zones. In (c), the signals can be simultaneously detected from a given zone of the plurality of zones.

In some embodiments, the collection area is dimensioned to accommodate the plurality of partitions in a single layer. In some embodiments, (b) is performed on the chip. In some embodiments, the wells are dimensioned to hold a single partition of the plurality of partitions.

An additional aspect of the disclosure provides a system for analyzing a nucleic acid sample of a subject. The system comprises a chip comprising a first channel and a second channel meeting at an intersection. During use, (1) the first channel directs an aqueous fluid comprising the nucleic acid sample and (2) the second channel directs a non-aqueous fluid towards the intersection, so as to form a plurality of partitions at the intersection upon contacting between the aqueous fluid and the non-aqueous fluid. Each of the plurality of partitions includes (i) the nucleic acid sample or portion thereof, and (ii) reagents necessary for nucleic acid amplification. The system also comprises one or more computer processors that are individually or collectively programmed to (i) subject the nucleic acid sample or portion thereof in each of the plurality of partitions to a nucleic acid amplification reaction under conditions that are sufficient to yield an amplification product(s) of the nucleic acid sample or portion thereof, and (ii) subsequent to (i), simultaneously detect signals indicative of a presence or absence of the amplification product(s) in the plurality of partitions while the plurality of partitions are immobilized by wells in a collection area. Each of the wells has a dimension that is less than an average diameter of a given partition of the plurality of partitions.

In another aspect, the disclosure provides a non-transitory computer-readable medium comprising machine executable code that, upon execution by one or more computer processors, implements a method for analyzing a nucleic acid sample of a subject. The method comprises (a) directing (1) an aqueous fluid comprising the nucleic acid sample through a first channel and (2) a non-aqueous fluid through a second channel towards a plurality of intersections in a chip, so as to form a plurality of partitions at the plurality of intersections upon contacting between the aqueous fluid and the non-aqueous fluid. Each of the plurality of partitions includes (i) the nucleic acid sample or portion thereof, and (ii) reagents necessary for nucleic acid amplification. The method also comprises (b) subjecting the nucleic acid sample or portion thereof in each of the plurality of partitions to a nucleic acid amplification reaction under conditions that are sufficient to yield an amplification product(s) of the nucleic acid sample or portion thereof; and (c) with the plurality of partitions disposed in a collection area downstream of the plurality of intersections, simultaneously detecting signals indicative of a presence or absence of the amplification product(s) in the plurality of partitions.

In another aspect, the disclosure provides a non-transitory computer-readable medium comprising machine executable code that, upon execution by one or more computer processors, implements a method for analyzing a nucleic acid sample of a subject. The method comprises (a) forming a plurality of partitions upon contact between an aqueous fluid comprising the nucleic acid sample and a non-aqueous fluid. Each of the plurality of partitions includes (i) the nucleic acid sample or portion thereof, and (ii) reagents necessary for nucleic acid amplification. The method also comprises (b) subjecting the nucleic acid sample or portion thereof in each of the plurality of partitions to a nucleic acid amplification reaction under conditions that are sufficient to yield an amplification product(s) of the nucleic acid sample or portion thereof; and (c) subsequent to (b), with the plurality of partitions disposed in a collection area that is substantially planar, simultaneously detecting signals indicative of a presence or absence of the amplification product(s) in the plurality of partitions.

An additional aspect of the disclosure provides a non-transitory computer-readable medium comprising machine executable code that, upon execution by one or more computer processors, implements a method for analyzing a nucleic acid sample of a subject. The method comprises (a) forming a plurality of partitions upon contact between an aqueous fluid comprising the nucleic acid sample and a non-aqueous fluid. Each of the plurality of partitions includes (i) the nucleic acid sample or portion thereof, and (ii) reagents necessary for nucleic acid amplification. The method also comprises (b) subjecting the nucleic acid sample or portion thereof in each of the plurality of partitions to a nucleic acid amplification reaction under conditions that are sufficient to yield an amplification product(s) of the nucleic acid sample or portion thereof; and (c) subsequent to (b), simultaneously detecting signals indicative of a presence or absence of the amplification product(s) in the plurality of partitions while the plurality of partitions are immobilized by wells in a collection area. Each of the wells has a dimension that is less than an average diameter of a given partition of the plurality of partitions.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a scheme of an example method of the present disclosure;

FIG. 2 illustrates a scheme of an example method of the present disclosure;

FIG. 3 illustrates an example chip of the present disclosure;

FIG. 4 (panels A and B) schematically illustrates an example chip of the present disclosure;

FIG. 5 demonstrates a side view of an example collection area of the present disclosure;

FIG. 6 illustrates an example detector as described in the present disclosure;

FIG. 7 demonstrates an example of detecting a signal according to the present disclosure;

FIG. 8 illustrates an example system of the present disclosure;

FIG. 9 shows an example computer control system that is programmed or otherwise configured to implement methods provided herein;

FIG. 10 (panels A and B) schematically illustrates an example droplet generation device that may be used with methods and systems of the present disclosure; and

FIG. 11 (panels A and B) schematically illustrates an example droplet generation device that may be used with methods and systems of the present disclosure.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a” “an” and “the” include plural references unless the context clearly dictates otherwise. For example, the term “a molecule” includes a plurality of molecules, including mixtures thereof.

As used herein, the terms “amplifying” and “amplification” are used interchangeably and generally refer to generating one or more copies or “amplified product” of a nucleic acid. The term “DNA amplification” generally refers to generating one or more copies of a DNA molecule or “amplified DNA product”. The term “reverse transcription amplification” generally refers to the generation of deoxyribonucleic acid (DNA) from a ribonucleic acid (RNA) template via the action of a reverse transcriptase.

As used herein, the term “cycle threshold” or “Ct” generally refers to the cycle during thermocycling in which an increase in a detectable signal due to amplified product reaches a statistically significant level above background signal.

As used herein, the terms “denaturing” and “denaturation” are used interchangeably and generally refer to the full or partial unwinding of the helical structure of a double-stranded nucleic acid, and in some cases the unwinding of the secondary structure of a single stranded nucleic acid. Denaturation may include the inactivation of the cell wall(s) of a pathogen or the shell of a virus, and the inactivation of the protein(s) of inhibitors. Conditions at which denaturation may occur include a “denaturation temperature” that generally refers to a temperature at which denaturation is permitted to occur and a “denaturation duration” that generally refers to an amount of time allotted for denaturation to occur.

As used herein, the term “elongation” generally refers to the incorporation of nucleotides to a nucleic acid in a template directed fashion. Elongation may occur via the aid of an enzyme, such as, for example, a polymerase or reverse transcriptase. Conditions at which elongation may occur include an “elongation temperature” that generally refers to a temperature at which elongation is permitted to occur and an “elongation duration” that generally refers to an amount of time allotted for elongation to occur.

As used herein, the term “nucleic acid” generally refers to a polymeric form of nucleotides of any length, either deoxyribonucleotides or ribonucleotides, or analogs thereof. Nucleotides may be nucleoside triphosphate, such as deoxyribonucleotide triphosphate (dNTP). Nucleic acids may have any three dimensional structure, and may perform any function, known or unknown. Non-limiting examples of nucleic acids include DNA, and RNA. Nucleic acids can include coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non nucleotide components. A nucleic acid may be further modified after polymerization, such as by conjugation or binding with a reporter agent.

As used herein, the term “primer extension reaction” generally refers to the denaturing of a double-stranded nucleic acid, binding of a primer to one or both strands of the denatured nucleic acid, followed by elongation of the primer(s).

As used herein, the term “reaction mixture” generally refers to a composition comprising reagents necessary to complete nucleic acid amplification (e.g., DNA amplification, RNA amplification), with non-limiting examples of such reagents that include primer sets having specificity for target RNA or target DNA, DNA produced from reverse transcription of RNA, a DNA polymerase, a reverse transcriptase (e.g., for reverse transcription of RNA), suitable buffers (including zwitterionic buffers), co-factors (e.g., divalent and monovalent cations), dNTPs, and other enzymes (e.g., uracil-DNA glycosylase (UNG)), etc). In some cases, reaction mixtures can also comprise one or more reporter agents.

As used herein, a “reporter agent” generally refers to a composition that yields a detectable signal, the presence or absence of which can be used to detect the presence of amplified product.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogues thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the term “subject” generally refers to an entity or a medium that has testable or detectable genetic information. A subject can be a person or individual. A subject can be a vertebrate, such as, for example, a mammal. Non-limiting examples of mammals include murines, simians, humans, farm animals, sport animals, and pets. Other examples of subjects include, for example, food, plant, soil, and water.

As used herein, the term “fluid” generally refers to a liquid or a gas. A fluid cannot maintain a defined shape and will flow during an observable time frame to fill the container in which it is put. Thus, the fluid may have any suitable viscosity that permits flow. If two or more fluids are present, each fluid may be independently selected among essentially any fluids (liquids, gases, and the like) by those of ordinary skill in the art.

As used herein, the term “aqueous fluid” generally refers to a fluid that is made with, of, or from water, or a fluid that contains water. For example, an aqueous fluid may be an aqueous solution with water as the solvent. An aqueous fluid of the present disclosure may comprise reagents necessary for conducting a desired chemical reaction, e.g., polymerase chain reaction (PCR). Non-limiting examples of aqueous fluid include, but are not limited to, water and other aqueous solutions comprising water, such as cell or biological media, ethanol, salt solutions, etc.

As used herein, the term “non-aqueous fluid” generally refers to a fluid that is made from, with, or using a liquid other than water. Non-limiting examples of non-aqueous fluid include, but are not limited to, oils such as hydrocarbons, silicon oils, fluorocarbon oils, organic solvents etc.

As used herein, the term “intersection” generally refers to a point or area, where one channel crosses or meets another channel.

As used herein, the term “partition” generally refers to a division into or distribution in portions or shares. Examples of partitions include droplets and wells.

As used herein, the term “droplet” generally refers to an isolated portion of a first fluid (e.g., an aqueous fluid) that is surrounded by a second fluid (e.g., a non-aqueous fluid). An emulsion may include a dispersion of droplets of a first fluid (e.g., liquid) in a second fluid. The first fluid may be immiscible in the second fluid. In some embodiments, the first fluid and the second fluid are substantially immiscible. A droplet of the present disclosure may be spherical or assume other shapes, such as, for example, shapes with elliptical cross-sections. The diameter of a droplet, in a non-spherical droplet, is the diameter of a perfect mathematical sphere having the same volume as the non-spherical droplet. A droplet of the present disclosure may be a single emulsion, a double emulsion, or a triple emulsion, etc.

A droplet may include a skin. The skin may form upon heating the droplet. The skin may have a higher viscosity than an interior of the droplet. In some cases, the skin may prevent the droplet from fusing with other droplets.

As used herein, the term “microfluidic” generally refers to a chip, area, device, article, or system including at least one fluid channel having a cross-sectional dimension of less than about 10 mm, 1 mm, 0.5 mm, or 0.1 mm.

As used herein, a “cross-sectional dimension” of a channel may be measured perpendicularly with respect to the general direction of fluid flow within the channel.

As used herein, the term “channel” generally refers to a feature on or in a device or substrate (e.g., a chip) that at least partially directs flow of a fluid. A channel may have any cross-sectional shape (circular, oval, triangular, irregular, square or rectangular, etc.) and may be covered or uncovered. When a channel is completely covered, at least one portion of the channel may have a cross-section that is completely enclosed, or the entire channel may be completely enclosed along its entire length with the exception of its inlets and/or outlets or openings. A channel of the present disclosure may be of any suitable length. The channel may be straight, substantially straight, or it may contain one or more curves, bends, etc. For example, the channel may have a serpentine or a spiral configuration. In some embodiments, the channel includes one or more branches, with some or all of which connected with one or more other channel(s). When a channel is curved or bended with a corner or a turning point, the corner or turning point may be rounded so that a fluid or a partition would not be trapped in the corner or at the turning point.

A channel may also have an aspect ratio (length to average cross-sectional dimension) of at least 2:1, at least about 3:1, at least about 4:1, at least about 5:1, at least about 6:1, at least about 8:1, at least about 10:1, at least about 15:1, at least about 20:1, at least about 30:1, at least about 40:1, at least about 50:1, at least about 60:1, at least about 70:1, at least about 80:1, at least about 90:1, at least about 100:1 or more. An open channel generally will include characteristics that facilitate control over fluid transport, e.g., structural characteristics (an elongated indentation) and/or physical or chemical characteristics (hydrophobicity vs. hydrophilicity) or other characteristics that can exert a force (e.g., a containing force) on a fluid. Non-limiting examples of force actuators that can produce suitable forces include piezo actuators, pressure valves, electrodes to apply AC electric fields etc. . . . . The fluid within the channel may partially or completely fill the channel. When an open channel is used, the fluid may be held within the channel, for example, using surface tension (i.e., a concave or convex meniscus).

The term “sample,” as used herein, generally refers to any sample containing or suspected of containing a nucleic acid molecule. For example, a subject sample can be a biological sample containing one or more nucleic acid molecules. The biological sample can be obtained (e.g., extracted or isolated) from a bodily sample of a subject that can be selected from blood (e.g., whole blood), plasma, serum, urine, saliva, mucosal excretions, sputum, stool and tears. The bodily sample can be a fluid or tissue sample (e.g., skin sample) of the subject. In some examples, the sample is obtained from a cell-free bodily fluid of the subject, such as whole blood. In such instance, the sample can include cell-free DNA and/or cell-free RNA. In some other examples, the sample is an environmental sample (e.g., soil, waste, ambient air and etc.), industrial sample (e.g., samples from any industrial processes), and food samples (e.g., dairy products, vegetable products, and meat products).

### Method for Analyzing Nucleic Acid Samples

In an aspect, the present disclosure provides a method for analyzing a nucleic acid sample of a subject. The method comprises (a) directing (1) an aqueous fluid comprising the nucleic acid sample through a first channel and (2) a non-aqueous fluid through a second channel towards a plurality of intersections in a chip, so as to form a plurality of partitions at the plurality of intersections upon contacting between the aqueous fluid and the non-aqueous fluid, wherein each of the plurality of partitions includes (i) the nucleic acid sample or portion thereof, and (ii) reagents necessary for nucleic acid amplification. In the second channel, the non-aqueous fluid may be substantially free of the sample and the reagents.

The method may further comprise (b) subjecting the nucleic acid sample or portion thereof in each of the plurality of partitions to a nucleic acid amplification reaction under conditions that are sufficient to yield an amplification product(s) of the nucleic acid sample or portion thereof. In some embodiments, (b) may be performed in the collection area. In some embodiments, (b) may be performed on the chip.

Operation (b) may comprise subjecting each of the plurality of partitions to thermal cycling. The thermal cycling may comprise cycling a temperature of each of the plurality of partitions between a first temperature and a second temperature that is greater than the first temperature. In some cases, the thermal cycling may comprise cycling a temperature of each of the plurality of partitions between more than two different temperatures.

The aqueous fluid may comprise a nucleic acid sample and reagents necessary for nucleic acid amplification.

In one aspect, the present disclosure provides a method for analyzing a nucleic acid sample of a subject, comprising (a) forming a plurality of partitions upon contact between an aqueous fluid comprising the nucleic acid sample and a non-aqueous fluid, wherein each of the plurality of partitions includes (i) the nucleic acid sample or portion thereof, and (ii) reagents necessary for nucleic acid amplification.

The method may further comprise (b) subjecting the nucleic acid sample or portion thereof in each of the plurality of partitions to a nucleic acid amplification reaction under conditions that are sufficient to yield an amplification product(s) of the nucleic acid sample or portion thereof.

The method may further comprise, subsequent to (b), (c) with the plurality of partitions disposed in a collection area that is substantially planar, simultaneously detecting signals indicative of a presence or absence of the amplification product(s) in the plurality of partitions.

In some embodiments, the method further comprises directing the plurality of partitions to the collection area.

In some embodiments, (b) is performed in the collection area.

The collection area may be included in the chip.

In some embodiments, the collection area includes a plurality of zones, and wherein in (c), the signals are simultaneously detected from a given zone of the plurality of zones.

The collection area may be dimensioned to accommodate the plurality of partitions in a single layer. In some cases, though, the collection area may be dimensioned to also accommodate the plurality of partitions in multiple layers.

In some embodiments, operation (b) is performed on the chip.

In one aspect, the present disclosure provides a method for analyzing a nucleic acid sample of a subject, comprising (a) forming a plurality of partitions upon contact between an aqueous fluid comprising the nucleic acid sample and a non-aqueous fluid, wherein each of the plurality of partitions includes (i) the nucleic acid sample or portion thereof, and (ii) reagents necessary for nucleic acid amplification.

The method may further comprise (b) subjecting the nucleic acid sample or portion thereof in each of the plurality of partitions to a nucleic acid amplification reaction under conditions that are sufficient to yield an amplification product(s) of the nucleic acid sample or portion thereof.

The method may further comprise, subsequent to (b), (c) simultaneously detecting signals indicative of a presence or absence of the amplification product(s) in the plurality of partitions while the plurality of partitions are immobilized by wells in a collection area, wherein each of the wells has a dimension (e.g., length, width, depth) that is less than an average diameter of a given partition of the plurality of partitions.

In some embodiment, the method further comprises directing the plurality of partitions to the collection area.

In some embodiments, (b) is performed in the collection area.

The collection area may be included in the chip.

In some embodiments, the collection area includes a plurality of zones, and wherein in (c), the signals are simultaneously detected from a given zone of the plurality of zones.

The collection area may be dimensioned to accommodate the plurality of partitions in a single layer. In some cases, though, the collection area may be dimensioned to also accommodate the plurality of partitions in multiple layers.

In some embodiments, operation (b) is performed on the chip

In some embodiments, the wells are dimensioned to hold a single partition of the plurality of partitions.

The nucleic acid sample may be any suitable biological sample of a subject. For example, the nucleic acid sample may be solid matter (e.g., biological tissue) or may be a fluid (e.g., a biological fluid). In general, a biological fluid can include any fluid associated with living organisms. Non-limiting examples of a nucleic acid sample include blood (or components of blood, e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, micropiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues.

The nucleic acid sample may be obtained from a subject in a variety of ways. Non-limiting examples of approaches to obtain a nucleic acid sample from a subject include accessing the circulatory system (e.g., intravenously or intra-arterially via a syringe or other needle), collecting a secreted biological sample (e.g., feces, urine, sputum, saliva, etc.), surgically (e.g., biopsy), swabbing (e.g., buccal swab, oropharyngeal swab), pipetting, and breathing. Moreover, a nucleic acid sample may be obtained from any anatomical part of a subject where a desired biological sample is located.

In some embodiments, the nucleic acid sample is from a genome of the subject. In some embodiments, the nucleic acid sample is a cell free nucleic acid sample. For example, the nucleic acid sample may be cell free deoxyribonucleic acid (DNA).

The nucleic acid sample may be obtained directly from the subject. A nucleic acid sample obtained directly from a subject may be a nucleic acid sample that has not been further processed after being obtained from the subject, with the exception of any approach used to collect the nucleic acid sample from the subject for further processing. For example, blood is obtained directly from a subject by accessing the subject's circulatory system, removing the blood from the subject (e.g., via a needle), and entering the removed blood into a receptacle. The receptacle may comprise reagents (e.g., anti-coagulants) such that the blood sample is useful for further analysis. In another example, a swab may be used to access epithelial cells on an oropharyngeal surface of the subject. After obtaining the nucleic acid sample from the subject, the swab containing the biological sample can be contacted with a fluid (e.g., a buffer) to collect the biological fluid from the swab. In some embodiments, the nucleic acid sample is obtained directly from the subject and provided in the first channel without sample purification and/or ribonucleic acid (RNA) extraction. For example, the RNA or DNA in a nucleic acid sample may not be extracted from the nucleic acid sample when providing the sample in the first channel and/or the aqueous fluid. Moreover, in some embodiments, a target nucleic acid (e.g., a target RNA or target DNA) present in a nucleic acid sample is not concentrated prior to providing the nucleic acid sample to the aqueous fluid and/or the first channel.

A variety of nucleic acid amplification reactions may be used to amplify a target nucleic acid in the nucleic acid sample and generate an amplified product. Moreover, amplification of a nucleic acid may linear, exponential, or a combination thereof. Non-limiting examples of nucleic acid amplification methods include reverse transcription, primer extension, polymerase chain reaction, ligase chain reaction, helicase-dependent amplification (e.g., amplification that is preceded by contacting the nucleic acid with a helicase), asymmetric amplification, rolling circle amplification, and multiple displacement amplification (MDA). In some embodiments, the amplified product may be DNA. In cases where a target RNA is amplified, DNA can be obtained by reverse transcription of the RNA and subsequent amplification of the DNA can be used to generate an amplified DNA product. The amplified DNA product may be indicative of the presence of the target RNA in the biological sample. In cases where DNA is amplified, any DNA amplification method may be employed. Non-limiting examples of DNA amplification methods include polymerase chain reaction (PCR), variants of PCR (e.g., real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, touchdown PCR), and ligase chain reaction (LCR). In some embodiments, DNA amplification is linear. In some embodiments, DNA amplification is exponential. In some embodiments, DNA amplification is achieved with nested PCR, which can improve sensitivity of detecting amplified DNA products.

In any of the various aspects, nucleic acid amplification reactions described herein may be conducted in parallel. In general, parallel amplification reactions are amplification reactions that occur in the same reaction partition (e.g., the same droplet) and at the same time. Parallel nucleic acid amplification reactions may be conducted, for example, by including reagents necessary for each nucleic acid amplification reaction in a partition to obtain a reaction mixture and subjecting the reaction mixture to conditions necessary for each nucleic amplification reaction. For example, reverse transcription amplification and DNA amplification may be conducted in parallel, by providing reagents necessary for both amplification methods in a partition to obtain a reaction mixture and subjecting the reaction mixture to conditions suitable for conducting both amplification reactions. DNA generated from reverse transcription of the RNA may be amplified in parallel to generate an amplified DNA product. Any suitable number of nucleic acid amplification reactions may be conducted in parallel. In some cases, at least 1, 2, 3, 4, 5, 6, 7, 8, 9, 10, 11, 12, 13, 14, 15, 16, 17, 18, 19, 20, 30, 40, 50, 100, 200, 300, 400, 500, 1000, 10,000, or more nucleic acid amplification reactions are conducted in parallel.

An advantage of conducting nucleic acid amplification reactions in parallel can include fast transitions between coupled nucleic acid amplification reactions. For example, a target nucleic acid (e.g., target RNA, target DNA) may be extracted or released from a biological sample during heating phases of parallel nucleic acid amplification. In the case of a target RNA, for example, the biological sample comprising the target RNA can be heated and the target RNA released from the biological sample. The released target RNA can immediately begin reverse transcription (via reverse transcription amplification) to produce complementary DNA. The complementary DNA can then be immediately amplified, often on the order of seconds. A short time between release of a target RNA from a biological sample and reverse transcription of the target RNA to complementary DNA may help minimize the effects of inhibitors in the biological sample that may impede reverse transcription and/or DNA amplification.

The reagents necessary for nucleic acid amplification may include a polymerizing enzyme and primers having sequence complementary with a target nucleic acid sequence.

In any of the various aspects, primers sets directed to a target nucleic acid may be utilized to conduct nucleic acid amplification reaction. Primer sets generally comprise one or more primers. For example, a primer set may comprise about 1, 2, 3, 4, 5, 6, 7, 8, 9, 10, 20, 30, 40, 50, or more primers. In some embodiments, a primer set comprises primers directed to different amplified products or different nucleic acid amplification reactions. For example, a primer set may comprise a first primer necessary to generate a first strand of nucleic acid product that is complementary to at least a portion of the target nucleic acid and a second primer complementary to the nucleic acid strand product necessary to generate a second strand of nucleic acid product that is complementary to at least a portion of the first strand of nucleic acid product.

For example, a primer set may be directed to a target RNA. The primer set may comprise a first primer that can be used to generate a first strand of nucleic acid product that is complementary to at least a portion the target RNA. In the case of a reverse transcription reaction, the first strand of nucleic acid product may be DNA. The primer set may also comprise a second primer that can be used to generate a second strand of nucleic acid product that is complementary to at least a portion of the first strand of nucleic acid product. In the case of a reverse transcription reaction conducted in parallel with DNA amplification, the second strand of nucleic acid product may be a strand of nucleic acid (e.g., DNA) product that is complementary to a strand of DNA generated from an RNA template.

Where desired, any suitable number of primer sets may be used. For example, about 1, 2, 3, 4, 5, 6, 7, 8, 9, 10, or more primer sets may be used. Where multiple primer sets are used, one or more primer sets may each correspond to a particular nucleic acid amplification reaction or amplified product.

In some embodiments, a DNA polymerase is used. Any suitable DNA polymerase may be used, including commercially available DNA polymerases. A DNA polymerase generally refers to an enzyme that is capable of incorporating nucleotides to a strand of DNA in a template bound fashion. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at a temperature from about 92° C. to 95° C. (e.g., 94° C. to 95° C.) for a time period from about 2 minutes to 10 minutes may be required, which may change the thermal profile based on different polymerases.

In some embodiments, a reverse transcriptase is used. Any suitable reverse transcriptase may be used. A reverse transcriptase generally refers to an enzyme that is capable of incorporating nucleotides to a strand of DNA, when bound to an RNA template. Non-limiting examples of reverse transcriptases include HIV-1 reverse transcriptase, M-MLV reverse transcriptase, AMV reverse transcriptase, telomerase reverse transcriptase, and variants, modified products and derivatives thereof.

The target nucleic acid sequence may be associated with a disease. The disease may be associated with a virus such as for example an RNA virus or a DNA virus. In some embodiments, the virus can be selected from the group consisting of human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, and Varicella virus. In some embodiments, the influenza virus is selected from the group consisting of H1N1 virus, H3N2 virus, H7N9 virus and H5N1 virus. In some embodiments, the adenovirus is adenovirus type 55 (ADV55) or adenovirus type 7 (ADV7). In some embodiments, the hepatitis C virus is armored RNA-hepatitis C virus (RNA-HCV). In some embodiments, the disease is associated with a pathogenic bacterium (e.g., Mycobacterium tuberculosis) or a pathogenic protozoan (e.g., Plasmodium).

In some embodiments, the disease is cancer. Non-limiting examples of the cancers include colorectal cancer, bladder cancer, ovarian cancer, testicular cancer, breast cancer, skin cancer, lung cancer, pancreatic cancer, stomach cancer, esophageal cancer, brain cancer, leukemia, liver cancer, endometrial cancer, prostate cancer, and head and neck cancer.

The target nucleic acid sequence may be associated with food safety. Food safety can be compromised by foodborne illness caused by pathogenic microbes. The pathogenic microbes may be bacteria, viruses, or parasites. Therefore, in some embodiments of the present disclosure, the target nucleic acid sequence is associated with a pathogenic bacterium, a pathogenic virus, or a pathogenic parasite that may compromise food safety.

In some embodiments, the food safety may be compromised by a pathogenic bacterium. Non-limiting examples of pathogenic bacteria include Campylobacter jejuni, Clostridium perfringens, Salmonella spp., Escherichia coli O157:H7 enterohemorrhagic (EHEC), Bacillus cereus, other virulent Escherichia coli such as enteroinvasive (EIEC), enteropathogenic (EPEC), enterotoxigenic (ETEC), enteroaggregative (EAEC or EAgEC), Listeria monocytogenes, Shigella spp., Staphylococcus aureus, Staphylococcal enteritis, Streptococcus, Vibrio cholerae, including O1 and non-O1, Vibrio parahaemolyticus, Vibrio vulnificus, Yersinia enterocolitica and Yersinia pseudotuberculosis, Brucella spp., Corynebacterium ulcerans, Coxiella burnetii or Q fever, Plesiomonas shigelloides, and the like. Sometimes the food safety is compromised by an enterotoxin secreted by a bacterium rather than the bacterium per se. Non-limiting examples of such enterotoxin-secreting bacteria include Staphylococcus aureus, Clostridium botulinum, Clostridium perfringens, Bacillus cereus, Pseudoalteromonas tetraodonis, Pseudomonas spp., Vibrio spp., and the like.

In some embodiments, the food safety may be compromised by a pathogenic virus. Non-limiting examples of pathogenic virus include Enterovirus, Hepatitis A, Hepatitis E, Norovirus, Rotavirus, and the like.

In some embodiments, the food safety may be compromised by a pathogenic parasite. Non-limiting examples of pathogenic parasite include Diphyllobothrium sp., Nanophyetus sp., Taenia saginata, Taenia soliurn, Fasciola hepatica, Anisakis sp., Ascaris lumbricoides, Eustrongylides sp., Trichinella spiralis, Trichuris trichiura, Acanthamoeba, Cryptosporidium parvum, Cyclospora cayetanensis, Entamoeba histolytica, Giardia lamblia, Sarcocystis hominis, Sarcocystis suihominis, Toxoplasma gondii, and the like.

The target nucleic acid sequence may be associated with prenatal testing. Prenatal testing may be conducted during gestation for detecting potential conditions, disorders or diseases associated with fetus. In some embodiments, the presence or the amount of the target nucleic acid sequence may be indicative of potential conditions, disorders or diseases in prenatal testing. Non-limiting conditions, disorders and diseases that may be detected in prenatal testing include spina bifida, cleft palate, Tay-Sachs disease, sickle cell anemia, thalassemia, cystic fibrosis, muscular dystrophy, fragile X syndrome, aneuploidy such as Down Syndrome (Trisomy 21), Edwards Syndrome (Trisomy 18), and Patau Syndrome (Trisomy 13), and the like.

The target nucleic acid sequence may be associated with genetic testing. Genetic testing may be conducted for various purposes, including, but not limited to detection of genetic disorders, forensic testing, molecular diagnosis, paternity/maternity testing, and the like. In some embodiments, the presence or the amount of the target nucleic acid sequence may be indicative of the result of a genetic testing.

The target nucleic acid sequence may be associated with cancer liquid biopsy. Cancer liquid biopsy is useful for detecting cancer by analyzing liquid samples from a subject (such as blood or bodily fluid) for indicators of cancers, such as circulating tumor cells or cell-free tumor nucleic acids. In some embodiments, the presence or the amount of the target nucleic acid sequence may be indicative of having cancer or being in the risk of having cancer in a cancer liquid biopsy. The cancer may be any cancer that can be diagnosed with a cancer liquid biopsy. Non-limiting examples of cancers that can be diagnosed with a cancer liquid biopsy include breast cancer, colon cancer, leukemia, lymphoma, stomach cancer, lung cancer, prostate cancer, and the like.

In some embodiments, the thermal cycling comprises a cycle of incubating a reaction mixture at a denaturation temperature for a denaturation duration and incubating a reaction mixture at an elongation temperature for an elongation duration.

Denaturation temperatures may vary depending upon, for example, the particular nucleic acid sample analyzed, the particular source of target nucleic acid (e.g., viral particle, bacteria) in the nucleic acid sample, the reagents used, and/or the desired reaction conditions. For example, a denaturation temperature may be from about 80° C. to about 110° C. In some examples, a denaturation temperature may be from about 90° C. to about 100° C. In some examples, a denaturation temperature may be from about 90° C. to about 97° C. In some examples, a denaturation temperature may be from about 92° C. to about 95° C. In still other examples, a denaturation temperature may be at least about 80°, at least about 81° C., at least about 82° C., at least about 83° C., at least about 84° C., at least about 85° C., at least about 86° C., at least about 87° C., at least about 88° C., at least about 89° C., at least about 90° C., at least about 91° C., at least about 92° C., at least about 93° C., at least about 94° C., at least about 95° C., at least about 96° C., at least about 97° C., at least about 98° C., at least about 99° C., or at least about 100° C.

Denaturation durations may vary depending upon, for example, the particular nucleic acid sample analyzed, the particular source of target nucleic acid (e.g., viral particle, bacteria) in the nucleic acid sample, the reagents used, and/or the desired reaction conditions. For example, a denaturation duration may be less than or equal to about 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, a denaturation duration may be no more than 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

Elongation temperatures may vary depending upon, for example, the particular nucleic acid sample analyzed, the particular source of target nucleic acid (e.g., viral particle, bacteria) in the nucleic acid sample, the reagents used, and/or the desired reaction conditions. For example, an elongation temperature may be from about 30° C. to about 80° C. In some examples, an elongation temperature may be from about 35° C. to about 72° C. In some examples, an elongation temperature may be from about 45° C. to about 65° C. In some examples, an elongation temperature may be from about 35° C. to about 65° C. In some examples, an elongation temperature may be from about 40° C. to about 60° C. In some examples, an elongation temperature may be from about 50° C. to about 60° C. In still other examples, an elongation temperature may be at least about 35°, 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

Elongation durations may vary depending upon, for example, the particular nucleic acid sample analyzed, the particular source of target nucleic acid (e.g., viral particle, bacteria) in the nucleic acid sample, the reagents used, and/or the desired reaction conditions. For example, an elongation duration may be less than or equal to about 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, an elongation duration may be no more than about 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

In any of the various aspects, multiple cycles of a primer extension reaction may be conducted. Any suitable number of cycles may be conducted. For example, the number of cycles conducted may be less than about 100, 90, 80, 70, 60, 50, 40, 30, 20, 10, or 5 cycles. The number of cycles conducted may depend upon, for example, the number of cycles necessary to obtain a detectable amplified product (e.g., a detectable amount of amplified DNA product that is indicative of the presence of a target RNA in a nucleic acid sample). For example, the number of cycles necessary to obtain a detectable amplified product (e.g., a detectable amount of DNA product that is indicative of the presence of a target RNA in a nucleic acid sample) may be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles.

The time for which amplification yields a detectable amount of amplified product indicative of the presence of a target nucleic acid amplified can vary depending upon the nucleic acid sample from which the target nucleic acid was obtained, the particular nucleic acid amplification reactions to be conducted, and the particular number of cycles of amplification reaction desired. For example, amplification of a target nucleic acid may yield a detectable amount of amplified product indicative to the presence of the target nucleic acid at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, a reaction mixture (e.g., within the partitions) is subjected to a plurality of series of primer extension reactions. An individual series of the plurality may comprise multiple cycles of a particular primer extension reaction, characterized, for example, by particular denaturation and elongation conditions as described elsewhere herein. Generally, each individual series differs from at least one other individual series in the plurality with respect to, for example, a denaturation condition and/or elongation condition. An individual series may differ from another individual series in a plurality of series, for example, with respect to any one, two, three, or all four of denaturing temperature, denaturing duration, elongation temperature, and elongation duration. Moreover, a plurality of series may comprise any number of individual series such as, for example, at least about or about 2, 3, 4, 5, 6, 7, 8, 9, 10, or more individual series.

For example, a plurality of series of primer extension reactions may comprise a first series and a second series. The first series, for example, may comprise more than ten cycles of a primer extension reaction, where each cycle of the first series comprises (i) incubating a reaction mixture (e.g., in the partitions) at about 92° C. to about 95° C. for no more than 30 seconds followed by (ii) incubating the reaction mixture at about 35° C. to about 65° C. for no more than about one minute. The second series, for example, may comprise more than ten cycles of a primer extension reaction, where each cycle of the second series comprises (i) incubating the reaction mixture (e.g., in the partitions) at about 92° C. to about 95° C. for no more than 30 seconds followed by (ii) incubating the reaction mixture at about 40° C. to about 60° C. for no more than about 1 minute. In this particular example, the first and second series differ in their elongation temperature condition. The example, however, is not meant to be limiting as any combination of different elongation and denaturing conditions could be used.

In some embodiments, the ramping time (i.e., the time the thermal cycler takes to transition from one temperature to another) and/or ramping rate is important for amplification. For example, the temperature and time for which amplification yields a detectable amount of amplified product indicative of the presence of a target nucleic acid can vary depending upon the ramping rate and/or ramping time. The ramping rate can impact the temperature(s) and time(s) used for amplification.

In some cases, the ramping time and/or ramping rate can be different between cycles. In some situations, however, the ramping time and/or ramping rate between cycles can be the same. The ramping time and/or ramping rate can be adjusted based on the sample(s) that are being processed.

In some situations, the ramping time between different temperatures can be determined, for example, based on the nature of the sample and the reaction conditions. The exact temperature and incubation time can also be determined based on the nature of the sample and the reaction conditions. In some embodiments, a single sample can be processed (e.g., subjected to amplification conditions) multiple times using multiple thermal cycles, with each thermal cycle differing for example by the ramping time, temperature, and/or incubation time. The best or optimum thermal cycle can then be chosen for that particular sample. This provides a robust and efficient method of tailoring the thermal cycles to the specific sample or combination of samples being tested.

In some embodiments, a target nucleic acid is subjected to a denaturing condition prior to initiation of a primer extension reaction. In the case of a plurality of series of primer extension reactions, the target nucleic acid may be subjected to a denaturing condition prior to executing the plurality of series or may be subjected to a denaturing condition between series of the plurality. For example, the target nucleic acid may be subjected to a denaturing condition between a first series and a second series of a plurality of series. Non-limiting examples of such denaturing conditions include a denaturing temperature profile (e.g., one or more denaturing temperatures) and a denaturing agent.

The partitions may include detectable moieties that permit detection of the signals. For example, the detectable moieties may yield a detectable signal whose presence or absence is indicative of the presence of an amplified product. The intensity of the detectable signal may be proportional to the amount of amplified product. In some cases, where amplified product is generated of a different type of nucleic acid than the target nucleic acid initially amplified, the intensity of the detectable signal may be proportional to the amount of target nucleic acid initially amplified. For example, in the case of amplifying a target RNA via parallel reverse transcription and amplification of the DNA obtained from reverse transcription, reagents necessary for both reactions may also comprise a detectable moiety that yield a detectable signal indicative of the presence of the amplified DNA product and/or the target RNA amplified. The intensity of the detectable signal may be proportional to the amount of the amplified DNA product and/or the original target RNA amplified. The use of a detectable moiety also enables real-time amplification methods, including real-time PCR for DNA amplification.

Detectable moieties may be linked with nucleic acids, including amplified products, by covalent or non-covalent interactions. Non-limiting examples of non-covalent interactions include ionic interactions, Van der Waals forces, hydrophobic interactions, hydrogen bonding, and combinations thereof. In some embodiments, detectable moieties bind to initial reactants and changes in detectable moiety levels are used to detect amplified product. In some embodiments, detectable moieties are only detectable (or non-detectable) as nucleic acid amplification progresses. In some embodiments, an optically-active dye (e.g., a fluorescent dye) is used as a detectable moiety. Non-limiting examples of dyes include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5- (or 6-) iodoacetamidofluorescein, 5-{[2(and 3)-5-(Acetylmercapto)-succinyl]amino}fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In some embodiments, a detectable moiety is a sequence-specific oligonucleotide probe that is optically active when hybridized with an amplified product. Due to sequence-specific binding of the probe to the amplified product, use of oligonucleotide probes can increase specificity and sensitivity of detection. A probe may be linked to any of the optically-active detectable moieties (e.g., dyes) described herein and may also include a quencher capable of blocking the optical activity of an associated dye. Non-limiting examples of probes that may be useful as detectable moieties include TaqMan probes, TaqMan Tamara probes, TaqMan MGB probes, or Lion probes, locked nucleic acid probes, and molecular beacons. Alternatively, the probe maybe any known probe that is useful in the context of the methods of the present disclosure.

In some embodiments and where a detectable moiety is an RNA oligonucleotide probe that includes an optically-active dye (e.g., fluorescent dye) and a quencher positioned adjacently on the probe. The close proximity of the dye with the quencher can block the optical activity of the dye. The probe may bind to a target sequence to be amplified. Upon the breakdown of the probe with the exonuclease activity of a DNA polymerase during amplification, the quencher and dye are separated, and the free dye regains its optical activity that can subsequently be detected.

In some embodiments, a detectable moiety is a molecular beacon. A molecular beacon includes, for example, a quencher linked at one end of an oligonucleotide in a hairpin conformation. At the other end of the oligonucleotide is an optically active dye, such as, for example, a fluorescent dye. In the hairpin configuration, the optically-active dye and quencher are brought in close enough proximity such that the quencher is capable of blocking the optical activity of the dye. Upon hybridizing with amplified product, however, the oligonucleotide assumes a linear conformation and hybridizes with a target sequence on the amplified product. Linearization of the oligonucleotide results in separation of the optically-active dye and quencher, such that the optical activity is restored and can be detected. The sequence specificity of the molecular beacon for a target sequence on the amplified product can improve specificity and sensitivity of detection.

In some embodiments, a detectable moiety is a radioactive species. Non-limiting examples of radioactive species include ¹⁴C, ¹²³I, ¹²⁴I, ¹²⁵I, ¹³¹I, Tc99m, ³⁵S, and ³H.

In some embodiments, a detectable moiety is an enzyme that is capable of generating a detectable signal. Detectable signal may be produced by activity of the enzyme with its substrate or a particular substrate in the case the enzyme has multiple substrates. Non-limiting examples of enzymes that may be used as detectable moieties include alkaline phosphatase, horseradish peroxidase, I²-galactosidase, alkaline phosphatase, β-galactosidase, acetylcholinesterase, and luciferase.

The non-aqueous fluid may comprise hydrophobic liquids. Non-limiting examples of the hydrophobic liquids include oils, such as hydrocarbons, silicon oils, fluorocarbon oils, organic solvents etc. In some embodiments, the oil is a fluorinated oil, such as such as HFE 7100, HFE 7500, FC-40, FC-43, FC-70, FC-3208, or a combination thereof. In some embodiments, the oil is a mineral oil, such as liquid paraffin, light mineral oil, white oil, refined mineral oil, cycloalkane oil, aromatic oil, or a combination thereof. The oil may also be any known oil that is useful for making droplets.

The non-aqueous fluid may comprise a surfactant. The surfactant may comprise a hydrophobic tail and a hydrophilic head group, a polymer-based tail and a hydrophilic head group, a polymer-based tail and a polymer-based head group, a fluorinated tail and a hydrophilic head group, or a fluorinated polymer-based tail and a hydrophilic polymer-based head group. In some embodiments, the surfactant is of a di-block copolymer or tri-block copolymer type. For example, the surfactant may be a block copolymer, such as a tri-block copolymer consisting of two perfluoropolyether blocks and one poly(ethylene)glycol block. In some embodiments, the surfactant is selected from the group consisting of PFPE-PEG-PFPE (perfluoropolyether-polyethylene glycol-perfluoropolyether), tri-block copolymer EA-surfactant (RainDance Technologies) and DMP (dimorpholino phosphate)-surfactant (Baret, Kleinschmidt, et al., 2009). The length of PEG in a polymeric species, including a polymeric surfactant, can have any suitable length and may vary between different polymeric species that can be used. The surfactant may be present in the non-aqueous fluid with a concentration of 0.0001% to 5% (w/w), e.g., 0.001% to 4% (w/w), 0.01% to 3% (w/w), 0.1% to 2% (w/w), 0.1% to 1% (w/w). In some embodiments, the surfactant is present in the non-aqueous fluid with a concentration of at least about, at most about or about 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.2% (w/w), 1.4% (w/w), 1.6% (w/w), 1.8% (w/w), 2.0% (w/w), 2.5% (w/w), 3.0% (w/w), 3.5% (w/w), 4.0% (w/w), 4.5% (w/w), 5.0% (w/w), 7.0% (w/w), 10.0% (w/w), 15.0% (w/w), 20.0% (w/w) or more or less.

The first channel for containing the aqueous fluid may be of any suitable length. In some embodiments, the first channel is substantially straight. In some embodiments, the first channel contains one or more curves, bends, etc. In some embodiments, the first channel has a serpentine or a spiral configuration. Moreover, in some embodiments, the first channel includes one or more branches, some or all of which may contain secondary channels connecting the main channel of the first channel with a second channel (or in some embodiments, more than one second channel). The first channel may also be connected to a source of fluid (e.g., an aqueous fluid), as discussed herein.

The length of the first channel may be measured to include regions of the first channel containing the secondary channels connecting the main channel of the first channel with one or more second channels, including branches of the first channel. For example, if the first channel has a “Y” or a “T” configuration, the total length of the first channel may include both branches, if both branches each contain secondary channels. In some embodiments, the total length of the first channel, containing the secondary channels, may be at least about 1 mm, at least about 2 mm, at least about 3 mm, at least about 5 mm, at least about 7 mm, at least about 1 cm, at least about 1.5 cm, at least about 2 cm, at least 2.5 cm, at least about 3 cm, at least about 5 cm, at least about 7 cm, at least about 10 cm, etc. In some embodiments, the total length of the first channel, containing the secondary channels, may be no more than about 10 cm, no more than about 7 cm, no more than about 5 cm, no more than about 3 cm, no more than about 2.5 cm, no more than about 2 cm, no more than about 1.5 cm, no more than about 1 cm, no more than about 7 mm, no more than about 5 mm, no more than about 3 mm, or no more than about 2 mm.

The cross-sectional area of the first channel may be substantially constant, or may vary. In some embodiments, the cross-sectional area of the first channel varies as a function of position in the direction of fluid flow within the first channel. The average cross-sectional area of the first channel may be, e.g., at least about 1,000 μm², at least about 2,000 μm², at least about 3,000 μm², at least about 5,000 μm², at least about 10,000 μm², at least about 20,000 μm², at least about 30,000 μm², at least about 50,000 μm², at least about 100,000 μm², at least about 200,000 μm², at least about 300,000 μm², at least about 500,000 μm², at least about 1,000,000 μm², or more. In some embodiments, the average cross-sectional area of the first channel is no more than about 1,000,000 μm², no more than about 500,000 μm², no more than about 300,000 μm², no more than about 200,000 μm², no more than about 100,000 μm², no more than about 50,000 μm², no more than about 30,000 μm², no more than about 20,000 μm², no more than about 10,000 μm², no more than about 5,000 μm², no more than about 3,000 μm², or no more than about 2,000 μm². Combinations of any of these areas are also possible.

The cross-sectional area of the first channel may vary, e.g., along with the length of the channel. In some embodiments, the first channel has a cross-sectional area that varies between about 75% and about 125%, between about 80% and about 120%, between about 90% and about 110%, between about 95% and about 105%, between about 97% and about 103%, or between about 99% and about 101% of the average cross-sectional area. In addition, the first channel may have any suitable cross-sectional shape, e.g., circular, oval, triangular, irregular, square, or rectangular etc.

The first channel may have any suitable maximal cross-sectional dimension. The maximal cross-sectional dimension generally refers to the largest dimension that can be contained within a cross-section of the first channel, where the cross-section is determined orthogonal to the direction of average fluid flow within the first channel. For example, the maximum cross-sectional dimension may be no more than 1 mm, no more than about 800 μm, no more than about 600 μm, no more than about 500 μm, no more than about 400 μm, no more than about 300 μm, no more than about 250 μm, no more than about 200 μm, no more than about 100 μm, no more than about 75 μm, no more than about 50 μm, no more than about 25 μm, no more than about 10 μm, etc. In some embodiments, the maximum cross-sectional dimension may be at least about 5 μm, at least about 10 μm, at least about 25 μm, at least about 50 μm, at least about 75 μm, at least about 100 μm, at least about 200 μm, at least about 250 μm, at least about 300 μm, at least about 400 μm, at least about 500 μm, at least about 600 μm, at least about 800 μm, etc.

The first channel may be in fluidic communication with one or more second channel(s). The second channel may be microfluidic. However, in some embodiments, one or both of the first and second channels is not microfluidic.

In some embodiments, the second channel is separated from the first channel by a relatively constant distance of separation, and/or the first channel and the second channel are substantially parallel to each other. In some embodiments, the first channel and the second channel have a distance of separation that is between about 75% and about 125% of the average distance of separation between the channels. The distance of separation may also vary between about 80% and about 120%, between about 90% and about 110%, between about 95% and about 105%, between about 97% and about 103%, or between about 99% and about 101%.

In some embodiments, more than one second channel may be present. Each of the second channels may be in fluidic communication with the first channel. If more than one second channel is present, each of the second channels may be at the same or different distances as the first channel. In addition, the second channels may have the same or different lengths, shapes, cross-sectional areas, or other properties. The second channels also may or may not be fluidly connected to each other.

A second channel may be of any suitable length. In some embodiments, the second channel is substantially straight. In some embodiments, the second channel contains one or more curves, bends, etc. In some embodiments, the shape of the second channel is substantially the same as the shape of the first channel, e.g., such that the second channel is separated from the first channel by a relatively constant distance of separation. In some embodiments, the second channel has a different shape.

A second channel may have any suitable length. In some embodiments, the length of the second channel is substantially the same as the first channel. In some embodiments, the length of the second channel is measured to include regions of the second channel containing the secondary channels connecting the main channel of the first channel with one or more second channels. In some embodiments, the total length of the second channel, containing the secondary channels, is at least about 1 mm, at least about 2 mm, at least about 3 mm, at least about 5 mm, at least about 7 mm, at least about 1 cm, at least about 1.5 cm, at least about 2 cm, at least 2.5 cm, at least about 3 cm, at least about 5 cm, at least about 7 cm, at least about 10 cm, etc. In some embodiments, the total length of the second channel, containing the secondary channels, is no more than about 10 cm, no more than about 7 cm, no more than about 5 cm, no more than about 3 cm, no more than about 2.5 cm, no more than about 2 cm, no more than about 1.5 cm, no more than about 1 cm, no more than about 7 mm, no more than about 5 mm, no more than about 3 mm, or no more than about 2 mm.

The cross-sectional area of the second channel may be substantially constant or may vary. In some embodiments, the cross-sectional area of the second channel may vary as a function of position in the direction of fluid flow within the second channel. In some embodiments, the average cross-sectional area of the second channel is at least about 1,000 μm², at least about 2,000 μm², at least about 3,000 μm², at least about 5,000 μm², at least about 10,000 μm², at least about 20,000 μm², at least about 30,000 μm², at least about 50,000 μm², at least about 100,000 μm², at least about 200,000 μm², at least about 300,000 μm², at least about 500,000 μm², at least about 1,000,000 μm² etc. In some embodiments, the average cross-sectional area of the second channel is no more than about 1,000,000 μm², no more than about 500,000 μm², no more than about 300,000 μm², no more than about 200,000 μm², no more than about 100,000 μm², no more than about 50,000 μm², no more than about 30,000 μm², no more than about 20,000 μm², no more than about 10,000 μm², no more than about 5,000 μm², no more than about 3,000 μm², or no more than about 2,000 μm².

In some embodiments, the cross-sectional area of the second channel may vary. For example, the cross-sectional area of the second channel may vary along with the length of the channel. In some embodiments, the second channel has a cross-sectional area that varies between about 75% and about 125%, between about 80% and about 120%, between about 90% and about 110%, between about 95% and about 105%, between about 97% and about 103%, or between about 99% and about 101% of the average cross-sectional area. The cross-sectional area of the second channel may be the same or different than the cross-sectional area of the first channel. In addition, the second channel may have any suitable cross-sectional shape, e.g., circular, oval, triangular, irregular, square, or rectangular, etc. The cross-sectional shape of the second channel may be the same or different than the cross-sectional shape of the first channel.

The second channel may have any suitable maximal cross-sectional dimension. The maximal cross-sectional dimension is the largest dimension that can be contained within a cross-section of the second channel, where the cross-section is determined orthogonal to the direction of average fluid flow within the second channel. For example, the maximum cross-sectional dimension may be no more than 1 mm, no more than about 800 μm, no more than about 600 μm, no more than about 500 μm, no more than about 400 μm, no more than about 300 μm, no more than about 250 μm, no more than about 200 μm, no more than about 100 μm, no more than about 75 μm, no more than about 50 μm, no more than about 25 μm, no more than about 10 μm, etc. In addition, in some embodiments, the maximum cross-sectional dimension is at least about 5 μm, at least about 10 μm, at least about 25 μm, at least about 50 μm, at least about 75 μm, at least about 100 μm, at least about 200 μm, at least about 250 μm, at least about 300 μm, at least about 400 μm, at least about 500 μm, at least about 600 μm, at least about 800 μm, etc. The maximal cross-sectional dimension of the second channel may be the same or different from the maximal cross-sectional dimension of the first channel.

The first channel may include a main channel and a plurality of secondary channels that intersect the second channel at a plurality of intersections. The aqueous fluid flowing from the main channel of the first channel may pass through one or more of the secondary channels to enter the non-aqueous fluid contained within the second channel. The aqueous fluid may be substantially immiscible with the non-aqueous fluid, and may thereby form droplets of aqueous fluid contained within the non-aqueous fluid. In some embodiments, the secondary channels are of substantially the same shape or size, and/or have a cross-sectional area that is substantially smaller than the cross-sectional area of the main channel or the second channel, such that the resistance to fluid flow is largely dominated by the dimensions of the secondary channels, which may result in the creation of substantially monodisperse droplets.

In some embodiments, the secondary channels may have an average resistance to fluid flow that is at least about 3 times greater than the resistance to fluid flow in the first and/or second channels. In addition, in certain embodiments, the average resistance to fluid flow in the secondary channels is at least about 5 times greater, at least about 10 times greater, at least about 20 times greater, at least about 30 times greater, at least about 50 times greater, at least about 75 times greater, at least about 100 times greater, at least about 200 times greater, at least about 300 times greater, at least about 500 times greater, or at least about 1,000 times greater than the resistance to fluid flow of the first and/or second channels. In some embodiments, the average resistance to fluid flow in the secondary channels is no more than about 1,000 times or 500 times greater than the resistance to fluid flow in the first and/or second channels. The secondary channels may have average resistances that are substantially the same. In some embodiments, the secondary channels have a resistance to fluid flow that varies between about 75% and about 125%, between about 80% and about 120%, between about 90% and about 110%, between about 95% and about 105%, between about 97% and about 103%, or between about 99% and about 101% of the average resistance to fluid flow of all of the secondary channels.

In some embodiments, a high resistance to fluid flow is created using a secondary channel having a relatively small cross-sectional area or a relatively small minimum or maximum cross-sectional dimension within the secondary channel. In addition, in some embodiments, high resistances is created using other techniques, such as coating the secondary channel and/or forming a relatively tortuous secondary channel, in addition or instead of controlling the cross-sectional area or cross-sectional dimension within the channel. Accordingly, the secondary channel may be substantially straight, or the secondary channel may contain one or more curves, bends, etc. If more than one secondary channel is present, the secondary channels may each have the same or different shapes. For example, some or all of the secondary channels may be substantially straight. In addition, a secondary channel may have any suitable cross-sectional shape, e.g., circular, oval, triangular, irregular, square or rectangular etc., and each secondary channel may independently have the same or different cross-sectional shapes. The cross-sectional shape of the secondary channels may be the same or different than the cross-sectional shape of the main channel and/or the second channel.

A secondary channel may have any suitable maximal cross-sectional dimension. The maximal cross-sectional dimension is the largest dimension that can be contained within a cross-section of the secondary channel, where the cross-section is determined orthogonal to the direction of average fluid flow within the secondary channel. For example, the maximum cross-sectional dimension may be no more than 1 mm, no more than about 800 μm, no more than about 600 μm, no more than about 500 μm, no more than about 400 μm, no more than about 300 μm, no more than about 250 μm, no more than about 200 μm, no more than about 100 μm, no more than about 75 μm, no more than about 50 μm, no more than about 25 μm, no more than about 10 μm, etc. In some embodiments, the maximum cross-sectional dimension is at least about 5 μm, at least about 10 μm, at least about 25 μm, at least about 50 μm, at least about 75 μm, at least about 100 μm, at least about 200 μm, at least about 250 μm, at least about 300 μm, at least about 400 μm, at least about 500 μm, at least about 600 μm, at least about 800 μm, etc. In addition, the height of a secondary channel may be the same or different than the height of a first or second channel.

In some embodiments, a secondary channel has a ratio of the smallest cross-sectional dimension to the largest cross-sectional dimension within the channel of at least about 1:1.1, at least about 1:1.5, at least about 1:2, at least about 1:3, at least about 1:5, at least about 1:7, at least about 1:10, at least about 1:15, at least about 1:20, at least about 1:25, at least about 1:30, at least about 1:35, at least about 1:40, at least about 1:50, at least about 1:60, at least about 1:70, at least about 1:80, at least about 1:90, at least about 1:100, etc. In some embodiments, the ratio is no more than about 1:100, no more than about 1:90, no more than about 1:80, no more than about 1:70, no more than about 1:60, no more than about 1:50, no more than about 1:40, no more than about 1:35, no more than about 1:30, no more than about 1:25, no more than about 1:20, no more than about 1:15, no more than about 1:10, no more than about 1:7, no more than about 1:5, no more than about 1:3, no more than about 1:2, no more than about 1:1.5, etc.

The secondary channel may have any suitable length. In some embodiments, the length of the secondary channel is determined by the distance of separation between the main channel and the second channel. In some embodiments, the secondary channels have an average length of at least about 10 μm, at least about 20 μm, at least about 30 μm, at least about 50 μm, at least about 100 μm, at least about 200 μm, at least about 300 μm, at least about 500 μm, at least about 1,000 μm, or at least about 2,000 μm etc. In some embodiments, the secondary channels have a length of no more than about 2,000 μm, no more than about 1,000 μm, no more than about 500 μm, no more than about 300 μm, no more than about 200 μm, no more than about 100 μm, no more than about 50 μm, no more than about 30 μm, no more than about 20 μm, or no more than about 10 μm. The lengths of the secondary channels may be substantially the same, or the lengths may vary between about 75% and about 125%, between about 80% and about 120%, between about 90% and about 110%, between about 95% and about 105%, between about 97% and about 103%, or between about 99% and about 101% of the average length of all of the secondary channels (or the distance of separation between the first and second channels).

In some embodiments, the average cross-sectional area of the secondary channels is at least about 20 μm², at least about 30 μm², at least about 50 μm², at least about 75 μm², at least about 100 μm², at least about 300 μm², at least about 400 μm², at least about 500 μm², at least about 750 μm², at least about 1,000 μm², at least about 1,600 μm², at least about 2,000 μm², at least about 3,000 μm², at least about 4,000 μm², at least about 5,000 μm², at least about 6,000 μm², at least about 6,400 μm², at least about 7,000 μm², at least about 8,000 μm², at least about 9,000 μm², at least about 10,000 μm², etc. In some embodiments, the average cross-sectional area of the secondary channels is no more than about 10,000 μm², no more than about 9,000 μm², no more than about 8,000 μm², no more than about 7,000 μm², no more than about 6,400 μm², no more than about 6,000 μm², no more than about 6,000 μm², no more than about 5,000 μm², no more than about 4,000 μm², no more than about 3,000 μm², no more than about 2,000 μm², no more than about 1,600 μm², no more than about 1,000 μm², no more than about 750 μm², no more than about 500 μm², no more than about 400 μm², no more than about 300 μm², no more than about 100 μm², no more than about 75 μm², no more than about 50 μm², no more than about 30 μm², no more than about 20 μm², etc.

In some embodiments, the secondary channel has a cross-sectional area that varies between about 75% and about 125%, between about 80% and about 120%, between about 90% and about 110%, between about 95% and about 105%, between about 97% and about 103%, or between about 99% and about 101% of the average cross-sectional area of all of the secondary channels. The cross-sectional area of a secondary channel may be substantially constant, or may vary. In some embodiments, the cross-sectional area of a secondary channel varies as a function of position in the direction of fluid flow within the secondary channel. In some embodiments, the secondary channel has a cross-sectional area that varies between about 75% and about 125%, between about 80% and about 120%, between about 90% and about 110%, between about 95% and about 105%, between about 97% and about 103%, or between about 99% and about 101% of the average cross-sectional area. In some embodiments, the volumes of the secondary channels are substantially the same. In some embodiments, the secondary channels have a volume that varies between about 75% and about 125%, between about 80% and about 120%, between about 90% and about 110%, between about 95% and about 105%, between about 97% and about 103%, or between about 99% and about 101% of the average volume of all of the secondary channels.

In some embodiments, the main channel and/or the second channel has a cross-sectional area at least about 10 times greater than the smallest cross-sectional area of the secondary channels, and in some embodiments, at least about 15 times greater, at least about 20 times greater, at least about 30 times greater, at least about 40 times greater, at least about 50 times greater, at least about 75 times greater, at least about 100 times greater, at least about 200 times greater, at least about 300 times greater, at least about 500 times greater, at least about 1,000 times greater, at least about 2,000 times greater, at least about 3,000 times greater, or at least about 5,000 times greater than the smallest cross-sectional area of the secondary channels. In some embodiments, the cross-sectional area of the main channel and/or the second channel is no more than about 5,000 times greater, no more than about 3,000 times greater, no more than about 2,000 times greater, no more than about 1,000 times greater, no more than about 500 times greater, no more than about 300 times greater, no more than about 200 times greater, no more than about 100 times greater, no more than about 75 times greater, no more than about 50 times greater, no more than about 40 times greater, no more than about 30 times greater, or no more than about 20 times greater than the smallest cross-sectional area of the secondary channels.

Any suitable number of secondary channels may be present. In some embodiments, larger numbers of secondary channels are useful in producing droplets at greater rates. In addition, if the resistance of the secondary channels to fluid flow is relatively large compared to the resistance of the first and/or second channels to fluid flow, then additional numbers of secondary channels may not substantially affect droplet production rates and/or the monodispersity of the droplets. Thus, in some embodiments, there are relatively large numbers of secondary channels connecting the main channel and the second channel. In some embodiments, there are at least 5, at least 10, at least 15, at least 20, at least 25, at least 30, at least 50, at least 75, at least 100, at least 200, at least 300, at least 400, at least 500, at least 600, at least 800, at least 1,000, at least 1,200, at least 1,500, at least 2,000, at least 2,500, etc. secondary channels connecting the main channel and the second channel.

The plurality of secondary channels may be oriented at any suitable angle with respect to the main channel and/or the second channel. In some embodiments, the angle of intersection between a secondary channel and a main channel and/or a second channel is about 90°. The secondary channels may each intersect the main channel and/or the second channel at substantially the same angle, or the intersection angles may each be independently the same or different. In addition, the angle of intersection with the main channel and with the second channel may also be the same or different. In some embodiments, the secondary channels each are oriented with respect to the main channel of the main channel and/or the second channel at an angle of between about 45° and about 135°, between about 45° and about 100°, between about 70° and about 110°, between about 80° and about 100°, between about 85° and about 95°, between about 88° and about 92°, etc. In some embodiments, a secondary channel joins the main channel of the main channel and/or the second channel at an angle of at least about 10°, about 15°, about 20°, about 25°, about 30°, about 35°, about 40°, about 45°, about 50°, about 55°, about 60°, about 65°, about 70°, about 75°, about 80°, about 85°, about 90°, about 95°, about 100°, about 105°, about 110°, about 115°, about 120°, about 125°, about 130°, about 135°, about 140°, about 145°, about 150°, about 155°, about 160°, about 165°, about 170°, or angles between any of these values (e.g., between about 90° and about 170°, etc.).

The secondary channels may be arrayed between the main channel and the second channel in any suitable arrangement. In some embodiments, the secondary channels are linearly periodically spaced, e.g., such that the distances between any of the secondary channel and its nearest neighboring secondary channel is substantially the same, or at least such that the distance of separation between any neighboring secondary channels is between about 75% and about 125%, between about 80% and about 120%, between about 90% and about 110%, between about 95% and about 105%, between about 97% and about 103%, or between about 99% and about 101% of the average distance of separation between neighboring secondary channels. In some embodiments, when the cross-sectional area of the secondary channels is substantially constant, the spacing between the secondary channels may be used to determine the size of the droplets.

In some embodiments, the secondary channels may be positioned relatively close to each other at the intersection of the secondary channels with the main channel and/or the second channels. For example, the secondary channels may be positioned such that the average distances between any of the secondary channel and its nearest neighboring secondary channel is substantially the same as the average cross-sectional area of the secondary channels. In some embodiments, the secondary channels are positioned to have a periodic spacing at the intersection of the secondary channels with the main channel and/or the second channel(s) that is between about 25% and about 400% of a smallest cross-sectional dimension of the secondary channels. In some embodiments, the periodic spacing is at least about 25%, at least about 50%, at least about 75%, at least about 100%, at least about 150%, or at least about 200% of the smallest cross-sectional dimension of the secondary channels, and/or the periodic spacing is no more than about 200%, no more than about 100%, no more than about 75%, or no more than about 50% of the smallest cross-sectional dimension of the secondary channels.

The chip may comprise multiple sets of the first channel, second channel, and plurality of intersections. For example, the chip may comprise 2, 3, 4, 5, 6, 7, 8, 9, 10 or more of the first channel and/or second channel. Each of the first channel may comprise a main channel and one or more secondary channel(s).

The plurality of intersections may be 2 or more, 3 or more, 4 or more, 5 or more, 10 or more, 15 or more, 20 or more, 25 or more, 30 or more, 35 or more, 40 or more, 50 or more, 60 or more, 70 or more, 80 or more, 90 or more, 100 or more intersections. An intersection may be formed by a main channel of a first channel with a second channel, by a secondary channel of a first channel with a second channel, and/or by a secondary channel of a first channel with a main channel of a first channel. The plurality of intersections may be arranged in a linear configuration or a non-linear configuration. For example, the plurality of intersections may be arranged in a 2-dimensional array of configuration. In addition, the plurality of intersections may be regularly or irregularly spaced.

A partition of the present disclosure may be an isolated portion of a first fluid (e.g., an aqueous solution) that is completely surrounded by a second fluid (e.g., a non-aqueous solution). In some embodiments, a partition is a droplet. A partition (e.g., a droplet) may be of any suitable shape and it may not necessarily be spherical. The diameter of a partition, in a non-spherical partition, is the diameter of a perfect mathematical sphere having the same volume as the non-spherical partition.

The plurality of partitions of the present disclosure may be formed when a portion of a first fluid (e.g., an aqueous fluid) is substantially surrounded by a second fluid (e.g., a non-aqueous fluid). As used herein, a portion of a first fluid is “surrounded” by a second fluid when a closed loop can be drawn around the first fluid through only the second fluid. A portion of a first fluid is “completely surrounded” by a second fluid if closed loops going through only the second fluid can be drawn around the first fluid regardless of direction. A portion of a first fluid is “substantially surrounded” by a second fluid if the loops going through only the second fluid can be drawn around the droplet depending on the direction.

As described elsewhere in the present disclosure, the plurality of partitions may be formed at one or more intersections formed by a main channel of a first channel, a second channel, and/or a secondary channel of a first channel. The plurality of partitions may be driven and/or pulled away from the plurality of intersections where they are formed by one or more forces, e.g., applied at one or more inlet and/or outlet of a channel. For example, a pump, gravity, capillary action, surface tension, electroosmosis, centrifugal forces, etc. may be used to drive and/or pull the partitions away from the plurality of intersections where they are formed. A vacuum (e.g., from a vacuum pump or other suitable vacuum source) may also be used. Non-limiting examples of pumps include syringe pumps, peristaltic pumps, pressurized fluid sources etc. In some embodiments, the plurality of partitions are formed at a plurality of intersections formed by a main channel of a first channel and a plurality of secondary channels of the first channel. In some embodiments, the plurality of intersections are located along the same side of the first channel.

The average size of the partitions (e.g., droplets) may depend on the properties (e.g. flow rate, viscosity) of one or more of the fluids, and/or the size, configuration, or geometry of the chip (e.g., the length and width of the channels, the spacing between adjacent channels, size of an orifice of a channel in an intersection, etc.).

A chip of the present disclosure may comprise the channels and the plurality of intersections, as described elsewhere in the disclosure. In some embodiments, one or more of the channels is microfluidic channel(s). In some embodiments, at least some of the channels are not microfluidic.

The chip may comprise any number of channels, including microfluidic channels. The channels may be arranged in any suitable configuration. The channels may be all interconnected, or there can be more than one network of channels present. The channels may independently be straight, curved, bent, etc. In some embodiments, the channels within a chip (when added together) have a total length of at least about 100 micrometers, at least about 300 micrometers, at least about 500 micrometers, at least about 1 mm, at least about 3 mm, at least about 5 mm, at least about 10 mm, at least about 30 mm, at least 50 mm, at least about 100 mm, at least about 300 mm, at least about 500 mm, at least about 1 m, at least about 2 m, or at least about 3 m. In some embodiments, a chip of the present disclosure has at least 2 channels, at least 3 channels, at least 4 channels, at least 5 channels, at least 10 channels, at least 20 channels, at least 30 channels, at least 40 channels, at least 50 channels, at least 60 channels, at least 70 channels, at least 80 channels, at least 90 channels, at least 100 channels, etc.

In some embodiments, at least some of the channels comprised in the chip are microfluidic channels. For example, some or all of the fluid channels in a chip can have a maximum cross-sectional dimension of less than about 2 mm, and in certain cases, less than about 1 mm. In some embodiments, all fluid channels in a chip are microfluidic and/or have a largest cross sectional dimension of no more than about 2 mm or about 1 mm. In some embodiments, the fluid channels are formed in part by a single component (e.g. an etched substrate or molded unit). Larger channels, tubes, chambers, reservoirs, etc. may be used to store fluids and/or deliver fluids to various elements or systems in the chip. In some embodiments, the maximum cross-sectional dimension of the channels in a chip is less than about 500 micrometers, less than about 200 micrometers, less than about 100 micrometers, less than about 50 micrometers, or less than about 25 micrometers.

In a chip, the channels may be arranged in any suitable configuration. Different channel arrangements may be used, for example, to manipulate fluids, partitions (e.g. droplets), and/or other species within the channels. For example, channels within the device may be arranged to generate partitions (e.g., discrete droplets, single emulsions, double emulsions or other multiple emulsions, etc.), to mix fluids and/or partitions or other species contained therein, to screen or sort fluids and/or partitions or other species contained therein, to split or divide fluids and/or partitions, to cause a reaction to occur (e.g., between two fluids, between a species carried by a first fluid and a second fluid, or between two species carried by two fluids to occur), etc.

Fluids may be delivered into channels within a chip via one or more fluid sources. Any suitable source of fluid can be used, and in some embodiments, more than one source of fluid is used. For example, a pump, gravity, capillary action, surface tension, electroosmosis, centrifugal forces, etc. may be used to deliver a fluid from a fluid source into one or more channels in the chip. A vacuum (e.g., from a vacuum pump or other suitable vacuum source) may also be used. Non-limiting examples of pumps include syringe pumps, peristaltic pumps, pressurized fluid sources etc. A fluid source may be a reservoir comprising the corresponding fluid, and the reservoir may be in fluid communication with one or more channels in the chip. A reservoir may comprise one or more outlets, the channels in the chip may comprise one or more inlets, at least one of the one or more outlets of the reservoir may be in fluid communication with at least one of the one or more inlets of the channels. In some embodiments, a hydrodynamic resistor (e.g., a valve, a filter, a sieve, a snaked-shaped channel etc.) may be comprised at an outlet of a reservoir and/or at an inlet of a channel to control fluid flow. In some embodiments, the hydrodynamic resistor comprises a snake-shaped channel with a total length of at least about 0.1 mm, 0.5 mm, 1 mm, 1.5 mm, 2 mm, 2.5 mm, etc. The snake-shaped channel may be folded back and forth for a few times, e.g., 2, 3, 4, 5, 6, 7, 8, or more times.

The chip may have any number of fluid sources associated with it, for example, 1, 2, 3, 4, 5, 6, 7, 8, 9, 10, etc., or more fluid sources. The fluid sources need not be used to deliver fluid into the same channel, e.g., a first fluid source may deliver a first fluid (an aqueous fluid) to a first channel while a second fluid source may deliver a second fluid (e.g., a non-aqueous fluid) to a second channel, etc. In some embodiments, two or more channels are arranged to intersect at one or more intersections. There may be any number of fluidic channel intersections within the chip, for example, 2, 3, 4, 5, 6, 7, 8, 9, 10, 50, 100, 150, 200, 300, 400, 500, 600, 700, 800, 900, 1000, or more intersections.

The method of the present disclosure may also comprise, (c) with the plurality of partitions disposed in a collection area downstream of the plurality of intersections, simultaneously detecting signals indicative of a presence or absence of the amplification product(s) in the plurality of partitions. In some cases, operation (c) may comprise simultaneously detecting signals indicative of a presence or absence of the amplification product(s) in all of the plurality of partitions. In some embodiments, the collection area includes a plurality of zones, and wherein in (c), the signals may be simultaneously detected from a given zone of the plurality of zones.

The method of the present disclosure may further comprise directing the plurality of partitions to the collection area. For example, a third channel may be employed for directing the plurality of partitions from the plurality of intersections to the collection area. The third channel may have a diameter that is greater than a cross-section of each of the plurality of partitions.

The third channel may connect one or more of the first and/or second channel(s) with the collection area. In some embodiments, a third channel may comprise a main channel and one or more secondary channel(s). The third channel may be of any suitable length. In some embodiments, the third channel is substantially straight. In some embodiments, the third channel contains one or more curves, bends, etc. In some embodiments, the third channel has a serpentine or a spiral configuration. Moreover, in some embodiments, the third channel includes one or more branches, some or all of which may contain secondary channels connecting the main channel of the third channel with the collection area.

The length of the third channel may be measured to include regions of the third channel containing the secondary channels connecting the main channel of the third channel with the collection area, including branches of the third channel. In some embodiments, the total length of the third channel, containing the secondary channels, may be at least about 1 mm, at least about 2 mm, at least about 3 mm, at least about 5 mm, at least about 7 mm, at least about 1 cm, at least about 1.5 cm, at least about 2 cm, at least 2.5 cm, at least about 3 cm, at least about 5 cm, at least about 7 cm, at least about 10 cm, etc. In some embodiments, the total length of the third channel, containing the secondary channels, may be no more than about 10 cm, no more than about 7 cm, no more than about 5 cm, no more than about 3 cm, no more than about 2.5 cm, no more than about 2 cm, no more than about 1.5 cm, no more than about 1 cm, no more than about 7 mm, no more than about 5 mm, no more than about 3 mm, or no more than about 2 mm.

The cross-sectional area of the third channel may be substantially constant, or may vary. In some embodiments, the cross-sectional area of the third channel varies as a function of position in the direction of fluid flow within the third channel. The average cross-sectional area of the third channel may be, e.g., at least about 1,000 μm², at least about 2,000 μm², at least about 3,000 μm², at least about 5,000 μm², at least about 10,000 μm², at least about 20,000 μm², at least about 30,000 μm², at least about 50,000 μm², at least about 100,000 μm², at least about 200,000 μm², at least about 300,000 μm², at least about 500,000 μm², at least about 1,000,000 μm², or more. In some embodiments, the average cross-sectional area of the third channel is no more than about 1,000,000 μm², no more than about 500,000 μm², no more than about 300,000 μm², no more than about 200,000 μm², no more than about 100,000 μm², no more than about 50,000 μm², no more than about 30,000 μm², no more than about 20,000 μm², no more than about 10,000 μm², no more than about 5,000 μm², no more than about 3,000 μm², or no more than about 2,000 μm².

The cross-sectional area of the third channel may vary, e.g., along with the length of the channel. In some embodiments, the third channel has a cross-sectional area that varies between about 75% and about 125%, between about 80% and about 120%, between about 90% and about 110%, between about 95% and about 105%, between about 97% and about 103%, or between about 99% and about 101% of the average cross-sectional area. In addition, the third channel may have any suitable cross-sectional shape, e.g., circular, oval, triangular, irregular, square, or rectangular etc.

The third channel may have any suitable maximal cross-sectional dimension. The maximal cross-sectional dimension generally refers to the largest dimension that can be contained within a cross-section of the third channel, where the cross-section is determined orthogonal to the direction of average fluid flow within the third channel. For example, the maximum cross-sectional dimension may be no more than 1 mm, no more than about 800 μm, no more than about 600 μm, no more than about 500 μm, no more than about 400 μm, no more than about 300 μm, no more than about 250 μm, no more than about 200 μm, no more than about 100 μm, no more than about 75 μm, no more than about 50 μm, no more than about 25 μm, no more than about 10 μm, etc. In some embodiments, the maximum cross-sectional dimension may be at least about 5 μm, at least about 10 μm, at least about 25 μm, at least about 50 μm, at least about 75 μm, at least about 100 μm, at least about 200 μm, at least about 250 μm, at least about 300 μm, at least about 400 μm, at least about 500 μm, at least about 600 μm, at least about 800 μm, etc. In some embodiments, the third channel has a diameter that is greater than a cross-section of each of the plurality of partitions.

The third channel may be in fluidic communication with one or more of the first channel, the second channel, and/or the collection area.

A collection area of the present disclosure may be included in the chip. In some embodiments, the collection area is provided separately from the chip. When the collection area is not included in the chip, it may be in fluidic communication with the chip. The plurality of partitions generated at the plurality of intersections may be directed to the collection area through one or more of the channels comprised in the chip and/or the collection area.

The collection area may have any suitable shape and/or configuration. For example, the collection area may be substantially planar. In some embodiments, the collection area is curvilinear, for example, the collection area may be circular. In some embodiments, the collection area is tilted. The collection area may be removable from the chip. In some embodiments, the collection area is rotatable.

In some embodiments, a plurality of chips may be comprised on one side of a rotatable support structure (e.g., a rotatable symmetric circular disk). Each of the plurality of chips may comprise the channels (e.g., the first channel and the second channel) for generating the plurality of partitions and the collection area. In some embodiments, the rotatable support structure comprises a plurality of collection areas (e.g., a collection area removed from a chip). Rotation of the support structure may be driven by a motor capable of adjusting the rotation speed. The plurality of chips or the plurality of collection areas may be positioned symmetrically with respect to the center of the support structure. In some embodiments, the plurality of chips or the plurality of collection areas are inserted or integrated in the rotatable support structure.

The collection area may be dimensioned to accommodate the plurality of partitions in a single layer. For example, the collection area may be dimensioned in a manner to avoid or have little to no stacking of the plurality of partitions. In some embodiments, the collection area is enclosed by two parallel planar surfaces, and the average distance between the two parallel planar surfaces defines a height of the collection area. In some embodiments the height of the collection area is about or less than about an average diameter of the partitions generated. For example, the height of the collection area may be less than about 2000 μm, less than about 1000 μm, less than about 750 μm, less than about 500 μm, less than about 400 μm, less than about 300 μm, less than about 200 μm, less than about 100 μm, less than about 90 μm, less than about 80 μm, less than about 70 μm, less than about 60 μm, less than about 50 μm, less than about 45 μm, less than about 40 μm, less than about 35 μm, less than about 30 μm, less than about 25 μm, less than about 20 μm, less than about 15 μm, less than about 10 μm, less than about 5 μm, less than about 1 μm, less than about 0.1 μm, less than about 0.01 μm or less. The collection area (or, when applicable, a planar surface comprised by the collection area) may have a diameter of at least or about 0.01 μm, 0.1 μm, 1 μm, 5 μm, 10 μm, 20 μm, 30 μm, 40 μm, 50 μm, 60 μm, 70 μm, 80 μm, 90 μm, 100 μm, 150 μm, 200 μm, 250 μm, 300 μm, 350 μm, 400 μm, 450 μm, 500 μm, 550 μm, 600 μm, 700 μm, 800 μm, 900 μm, 1 mm, 2 mm, 3 mm, 4 mm, 5 mm, 6 mm, 7 mm, 8 mm, 9 mm, 10 mm, or more etc. In some cases, though, the collection area may be dimensioned to also accommodate the plurality of partitions in multiple layers.

The collection area may comprise wells that are dimensioned to hold a single partition of the plurality of partitions. Each of the wells may have a dimension (e.g., width, length, depth) that is less than an average diameter of a given partition of the plurality of partitions. For example, each of the wells may have a dimension that is less than about 500 μm, less than about 400 μm, less than about 300 μm, less than about 200 μm, less than about 100 μm, less than about 90 μm, less than about 80 μm, less than about 70 μm, less than about 60 μm, less than about 50 μm, less than about 45 μm, less than about 40 μm, less than about 35 μm, less than about 30 μm, less than about 25 μm, less than about 20 μm, less than about 15 μm, or less than about 10 μm, or less.

A well may hold a whole droplet or a portion of a single droplet. As an alternative, a well may hold multiple droplets, such as at least 2, 3, 4, 5 or 10 droplets or more.

In some embodiments, at the collection area, each of the plurality of partitions is at an individually addressable location. For example, each of the plurality of partitions may be directed to a confined structure or space that is coded, arranged or arrayed in a way to enable identification of a partition present in any of such confined structures or spaces. In some embodiments, a confined structure or space may be a well dimensioned to accommodate a single partition.

Each of the plurality of partitions may be subjected to thermal cycling using a source of thermal energy that is external to the chip. For example, the source of thermal energy may be an infrared energy source. In some embodiments, each of the plurality of partitions may be subjected to thermal cycling using a source of thermal energy that is integrated with the chip. For example, the source of thermal energy is a thermoelectric element (e.g., a Peltier element) or a resistive heating element. Alternatively, the source of thermal energy may be an induction heating element.

In some embodiments, a thermoelectric element (e.g., a Peltier element) is attached to one side (e.g., the lower side) of the collection area, the thermoelectric element may be in close contact with the collection area and may have an area that is large enough to cover at least the entire collection area.

In some embodiments, one side of the collection area (e.g., the bottom side) may be made of heat-absorbing material capable of converting light energy into heat. For example, the side of the collection area opposing to the side made of heat-absorbing material (e.g., the top side) may be made of material able to transmit light (e.g., transparent material). Accordingly, light (e.g., emitted from a infra-red (IR) lamp positioned above the collection area) may get through and be used to elevate the temperature in the collection area (e.g., to change the temperature in the partitions).

In some embodiments, one or more temperature sensors may be comprised in the collection area to monitor temperatures e.g. in real-time. The temperature sensors may provide feedback information to a system controlling the energy source (e.g., the IR lamps). The control system receiving information from the temperature sensor may in turn be controlled by one or more computer processors that are individually or collectively programmed to adjust the energy source.

In cases where a plurality of chips or collections areas are comprised on or integrated in a rotatable support structure, as described elsewhere in the present disclosure, light sources (e.g., one or more infra-red (IR) lamps) may be positioned above one portion or above symmetrically located portions of the support structure. Thus, the plurality of chips or collection areas may be equally/evenly exposed to the energy source (e.g., light source) with the rotation of the support structure, which in turn makes sure that different chips or collection areas on/in the supporting structure are heated uniformly.

The chip may comprise one or more inlets and one or more outlets. Each inlet may be in fluid communication with one or more reservoir(s). A reservoir may be filled with a fluid (an aqueous fluid or a non-aqueous fluid) to be supplied in one or more of the channels comprised in the chip. An outlet may be located at one end of a channel comprised in the chip and/or the collection area. One or more partitions, a fluid, or a waste material may be driven to flow through an outlet to a reservoir that is in fluid communication with the outlet. In some embodiments, the inlet and/or outlet further comprises a soft ring (e.g., a rubber ring) or a connector (e.g., a rubber tube) to form sealed connections between the inlet/outlet and the reservoir(s). In some cases, the aqueous fluid and non-aqueous fluid may be subjected to flow using a pressure drop between the first channel and/or second channel, and the outlet, that is at least about 0.1 psi, at least about 0.5 psi, at least about 1 psi, at least about 5 psi, at least about 10 psi, at least about 15 psi, at least about 20 psi, at least about 30 psi, at least about 40 psi, at least about 50 psi, at least about 60 psi, at least about 70 psi, at least about 80 psi, at least about 90 psi, at least about 100 psi, at least about 150 psi, at least about 200 psi, at least about 250 psi, at least about 300 psi, at least about 350 psi, at least about 400 psi, at least about 450 psi, at least about 500 psi, at least about 750 psi or more. In some cases, the aqueous fluid and non-aqueous fluid may be subjected to flow using a pressure drop between the first channel and/or second channel, and the outlet, that is at most about 750 psi, at least about 500 psi, at least about 450 psi, at least about 400 psi, at least about 350 psi, at least about 300 psi, at least about 250 psi, at least about 200 psi, at least about 150 psi, at least about 100 psi, at least about 90 psi, at least about 80 psi, at least about 70 psi, at least about 60 psi, at least about 50 psi, at least about 40 psi, at least about 30 psi, at least about 20 psi, at least about 15 psi, at least about 10 psi, at least about 5 psi, at least about 1 psi, at least about 0.5 psi, at least about 0.1 psi or less. In some embodiments, by controlling the overall pressure drop between the inlet, the first channel and/or second channel, and the outlet to be substantially constant, a plurality of substantially monodisperse partitions are produced.

The chip and/or one or more of the reservoirs may comprise a filter or an enrichment device to remove undesired substances from the nucleic acid sample, and/or to enrich desired components in the nucleic acid sample. Non-limiting examples of the filter or enrichment device include filtration membranes, e.g., nitrocellulose, cellulose acetate, polycarbonate, polypropylene and polyvinylidene fluoride microporous membranes, and ultrafiltration membranes (e.g., those made from polysulfone, polyvinylidene fluoride, cellulose etc.). In some embodiments, the aqueous and/or non-aqueous fluid flowed from one or more of the reservoirs is driven through one or more of the filters to enter the inlet(s) of the chip. In some embodiments, filtrates are collected in a separate reservoir.

To simultaneously detect signals indicative of a presence or absence of the amplification product(s) in the plurality of partitions, the operation (c) may further comprise directing excitation energy to the plurality of partitions and detecting the signals as emissions from the plurality of partitions. The signals may be detected using a detector that is integrated with the chip. In some cases, the signals may be detected using a detector that is external to the chip. For example, the detector may be a charge-coupled device (CCD) camera.

The excitation energy may be provided by a source of excitation energy that is integrated with the chip. In some cases, the excitation energy may be provided by a source of excitation energy that is external to the chip. For example, the excitation energy may be provided by a light-emitting diode or a laser. The signals may be optical signals (e.g., fluorescent signals), electrochemical signals, and/or electrostatic signals. In some embodiments, on one side of the chip (e.g., above the collection area), an optical image acquisition device (e.g., a CCD camera) and accompanying fluorescence excitation light sources are provided.

In operation (c), the plurality of partitions may be flowing at a flow rate less than about 10 ml/h through the collection area, e.g., less than about 9 ml/h, less than about 8 ml/h, less than about 7 ml/h, less than about 6 ml/h, less than about 5 ml/h, less than about 4 ml/h, less than about 3 ml/h, less than about 2 ml/h, less than about 1 ml/h, less than about 0.5 ml/h, 0.1 ml/h, 0.01 ml/h or less etc. In some embodiments, in operation (c), the plurality of partitions is substantially stationary.

The method of the present disclosure may further comprise, subsequent to operation (c) (e.g., after detection of signals indicative of a presence or absence of the amplification product(s)), directing the plurality of partitions out of the collection area towards an outlet. The outlet may be under negative pressure. In some cases, the negative pressure may be less than or about −5 bar, −4 bar, −3 bar, −2 bar, −1 bar, −0.9 bar, −0.8 bar, −0.7 bar, −0.6 bar, −0.5 bar, −0.4 bar, −0.3 bar, −0.2 bar, −0.1 bar, −0.05 bar, −0.04 bar, −0.03 bar, −0.02 bar, −0.01 bar, −0.005 bar, −0.001 bar, −0.0001 bar, or less. The first channel and/or second channel may be under positive pressure with respect to the outlet. In some embodiments, the aqueous fluid, non-aqueous fluid and/or the partitions (e.g., droplets) is subjected to flow using a pressure drop between the first channel and/or second channel, and the outlet, that is at least about that is at least about 0.1 psi, at least about 0.5 psi, at least about 1 psi, at least about 5 psi, at least about 10 psi, at least about 15 psi, at least about 20 psi, at least about 30 psi, at least about 40 psi, at least about 50 psi, at least about 60 psi, at least about 70 psi, at least about 80 psi, at least about 90 psi, at least about 100 psi, at least about 150 psi, at least about 200 psi, at least about 250 psi, at least about 300 psi, at least about 350 psi, at least about 400 psi, at least about 450 psi, at least about 500 psi, at least about 750 psi or more. In some cases, the aqueous fluid and non-aqueous fluid may be subjected to flow using a pressure drop between the first channel and/or second channel, and the outlet, that is at most about 750 psi, at least about 500 psi, at least about 450 psi, at least about 400 psi, at least about 350 psi, at least about 300 psi, at least about 250 psi, at least about 200 psi, at least about 150 psi, at least about 100 psi, at least about 90 psi, at least about 80 psi, at least about 70 psi, at least about 60 psi, at least about 50 psi, at least about 40 psi, at least about 30 psi, at least about 20 psi, at least about 15 psi, at least about 10 psi, at least about 5 psi, at least about 1 psi, at least about 0.5 psi, at least about 0.1 psi or less.

The amplification product may be detected at a sensitivity of at least about 90%. For example, the amplification product may be detected at a sensitivity of at least 60%, at least 70%, at least 80%, at least 85%, at least 90%, at least 95%, at least 96%, at least 97%, at least 98%, at least 99% or higher. As used herein, sensitivity generally refers to a measure of the proportion of positive signals that are correctly identified as such.

The amplification product may be detected at a specificity of at least about 90%. For example, the amplification product may be detected at a specificity of at least 60%, at least 70%, at least 80%, at least 85%, at least 90%, at least 95%, at least 96%, at least 97%, at least 98%, at least 99% or higher. As used herein, specificity generally refers to a measure of the proportion of negatives signals that are correctly identified as such.

The chip or a component thereof (e.g., the channels, the collection area etc.) may be made with a variety of materials and methods. For example, the chip or a component thereof may be formed from solid materials, in which the channels may be formed via micromachining, film deposition processes such as spin coating and chemical vapor deposition, physical vapor deposition, laser fabrication, photolithographic techniques, etching methods including wet chemical or plasma processes, electrodeposition etc. Various fabrication processes (e.g., soft lithography, hot embossing, injection molding, and laser ablation) may be used to produce the chips or components thereof.

In some embodiments, the chip or a component thereof is formed of a polymer, for example, an elastomeric polymer such as polydimethylsiloxane (“PDMS”), polytetrafluoroethylene (“PTFE” or Teflon®), etc. For example, a channel (e.g., a microfluidic channel) may be implemented by fabricating the fluidic system separately using PDMS or other soft lithography techniques. Other examples of potentially suitable polymers include, but are not limited to, polyethylene terephthalate (PET), polyacrylate, polymethacrylate, polycarbonate, polystyrene, polyethylene, polypropylene, polyvinylchloride, cyclic olefin copolymer (COC), polytetrafluoroethylene, a fluorinated polymer, a silicone such as polydimethylsiloxane, polyvinylidene chloride, bis-benzocyclobutene (BCB), a polyimide, a fluorinated derivative of a polyimide, etc. Combinations, copolymers, or blends involving polymers including those described above are also envisioned.

In some embodiments, the chip or a component thereof is made from polymeric and/or flexible and/or elastomeric materials, and can be conveniently formed of a hardenable fluid, facilitating fabrication via molding (e.g. replica molding, injection molding, cast molding, etc.). The hardenable fluid can be essentially any fluid that can be induced to solidify, or that spontaneously solidifies, into a solid capable of containing and/or transporting fluids contemplated for use in and with a fluidic network. In one embodiment, the hardenable fluid comprises a polymeric liquid or a liquid polymeric precursor (i.e. a “prepolymer”). Suitable polymeric liquids include, for example, thermoplastic polymers, thermoset polymers, waxes, metals, or mixtures or composites thereof heated above their melting point. In some embodiments, a suitable polymeric liquid includes a solution of one or more polymers in a suitable solvent, which solution forms a solid polymeric material upon removal of the solvent, for example, by evaporation. Such polymeric materials can be solidified from, for example, a melt state or by solvent evaporation. A variety of polymeric materials, many of which are elastomeric, are suitable, and are also suitable for forming molds or mold masters, for embodiments where one or both of the mold masters is composed of an elastomeric material. Non-limiting examples of such polymers include polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A may be used, in addition to compounds based on aromatic amine, triazine, and cycloaliphatic backbones. Another example is Novolac polymers. Non-limiting examples of silicone elastomers suitable for use herein include those formed from precursors including the chlorosilanes such as methylchlorosilanes, ethylchlorosilanes, phenylchlorosilanes, etc. In some embodiments, silicone polymers (e.g., the silicone elastomer polydimethylsiloxane) are used. Non-limiting examples of PDMS polymers include those sold under the trademark Sylgard by Dow Chemical Co., Midland, Mich., e.g., Sylgard 182, Sylgard 184, and Sylgard 186.

One advantage of forming structures such as microfluidic structures or channels from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus, structures can be fabricated and then oxidized and essentially irreversibly sealed to other silicone polymer surfaces, or to the surfaces of other substrates reactive with the oxidized silicone polymer surfaces, without the need for separate adhesives or other sealing approaches. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma).

In some embodiments, the chip or a component thereof is produced using more than one layer or substrate, e.g., more than one layer of PDMS. For instance, chips having channels with multiple heights and/or chips having interfaces positioned such as described herein may be produced using more than one layer or substrate, which may then be assembled or bonded together, e.g., using plasma bonding, to produce the final chip. For example, a chip of the present disclosure may be molded from masters comprising two or more layers of photoresists, e.g., where two PDMS molds are then bonded together by activating the PDMS surfaces using O₂ plasma or other suitable techniques. For example, the masters from which the PDMS chip is cast may contain one or more layers of photoresist, e.g., to form a 3D chip. In some embodiments, one or more of the layers has one or more mating protrusions and/or indentations which are aligned to properly align the layers, e.g., in a lock-and-key fashion. For example, a first layer may have a protrusion (having any suitable shape) and a second layer may have a corresponding indentation which can receive the protrusion, thereby causing the two layers to become properly aligned with respect to each other.

One or more sides (e.g., walls) or portions of a channel may be coated, e.g., with a coating material, including photoactive coating materials. For example, in some embodiments, each of the microfluidic channels has substantially the same hydrophobicity. In other embodiments, various channels have different hydrophobicity. For example a first channel (or set of channels) at a common intersection may exhibit a first hydrophobicity, while the other channels may exhibit a second hydrophobicity different from the first hydrophobicity, e.g., exhibiting a hydrophobicity that is greater or less than the first hydrophobicity. In some embodiments, the channels are coated with sol-gel coatings. Other non-limiting examples of coatings include polymers, metals, or ceramic coatings etc.

Thus, some or all of the channels may be coated, or otherwise treated such that some or all of the channels, including the inlet and any secondary channel, each have substantially the same hydrophilicity. The coating materials may be used to control and/or alter the hydrophobicity of the wall of a channel. In some embodiments, a sol-gel is provided that can be formed as a coating on a substrate such as the wall of a channel such as a microfluidic channel. One or more portions of the sol-gel may be reacted to alter its hydrophobicity. For example, a portion of the sol-gel may be exposed to light, such as ultraviolet light, which may be used to induce a chemical reaction in the sol-gel that alters its hydrophobicity. The sol-gel may include a photoinitiator which, upon exposure to light, produces radicals. In some embodiments, the photoinitiator is conjugated to a silane or other material within the sol-gel. The radicals so produced may be used to cause a condensation or polymerization reaction to occur on the surface of the sol-gel, thus altering the hydrophobicity of the surface. Various portions of the chip or a channel may be reacted or left unreacted, e.g., by controlling exposure to light (e.g., using a mask).

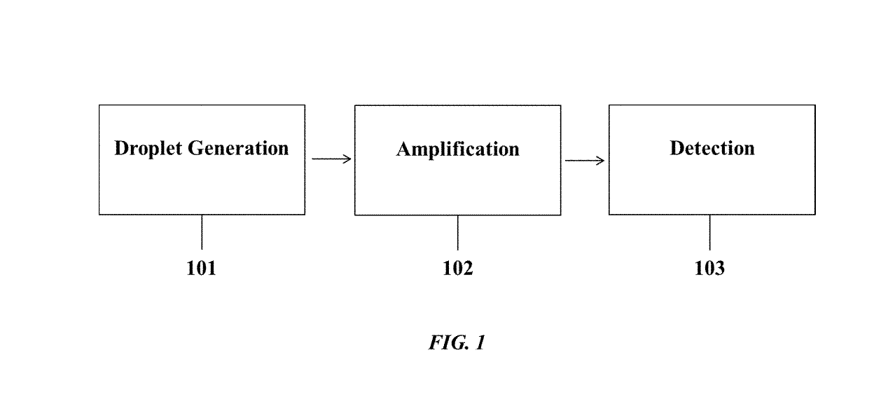

FIG. 1 illustrates an example of the method described in the present disclosure. Droplets comprising a nucleic acid sample to be analyzed may be generated in a first operation 101, and then, amplification of target nucleic acid molecules in the sample may be conducted in a second operation 102, afterwards, signals indicating presence or absence of the amplification product may be detected in a third operation 103. In some embodiments, droplet generation 101, nucleic acid amplification 102 and signal detection 103 are integrated in a single chip, and transportation or transfer of samples (e.g., droplets comprising samples to be analyzed) from one device (e.g., a droplet generator) to another device (e.g., a thermal cycler) is avoided. In some cases, droplet generation 101 is as described in WO/2014186440 (“APPARATUS AND METHOD FOR THE RAPID PRODUCTION OF DROPLETS”), which is entirely incorporated herein by reference.

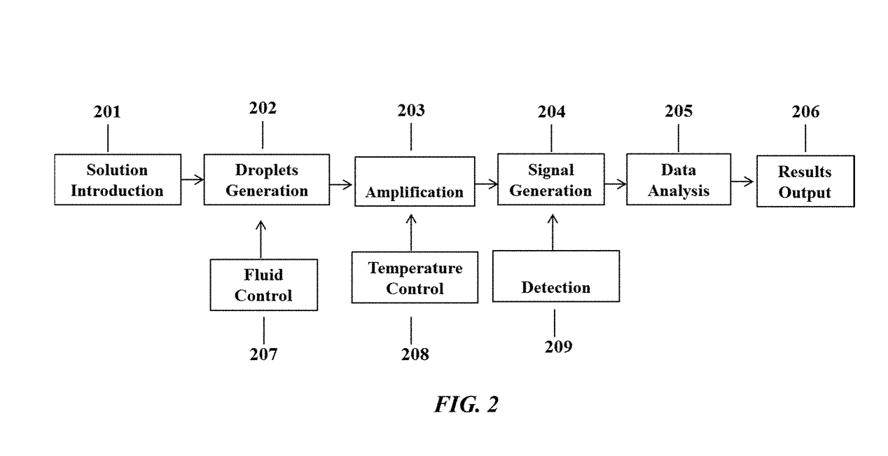

This is further illustrated by the process shown in FIG. 2. In an example, solutions (e.g., an aqueous solution comprising a nucleic acid sample and a non-aqueous solution) are introduced 201 to a chip of the present disclosure from one or more inlets; a first fluid (e.g., the aqueous fluid) is directed through a first channel and a second fluid (e.g., the non-aqueous fluid) is directed through a second channel towards a plurality of intersections in the chip, so as to generate 202 a plurality of droplets at the plurality of intersections upon contacting between the aqueous fluid and the non-aqueous fluid, this droplet generation operation 202 may be regulated by a fluid control operation 207. Then, the nucleic acid sample comprised in each of the droplets is subjected to a nucleic acid amplification reaction 203 under conditions sufficient to yield an amplification product(s) of the nucleic acid sample or portion thereof, the amplification process may be controlled by a temperature control operation 208 (e.g., as in a thermal cycler). Subsequent to nucleic acid amplification, signals indicating presence or absence of an amplification product may be generated 204, and the signals may be detected with a detection operation 209. The detection results may then be generated as data for data analysis 205, which then results in generation of a report as results output 206.

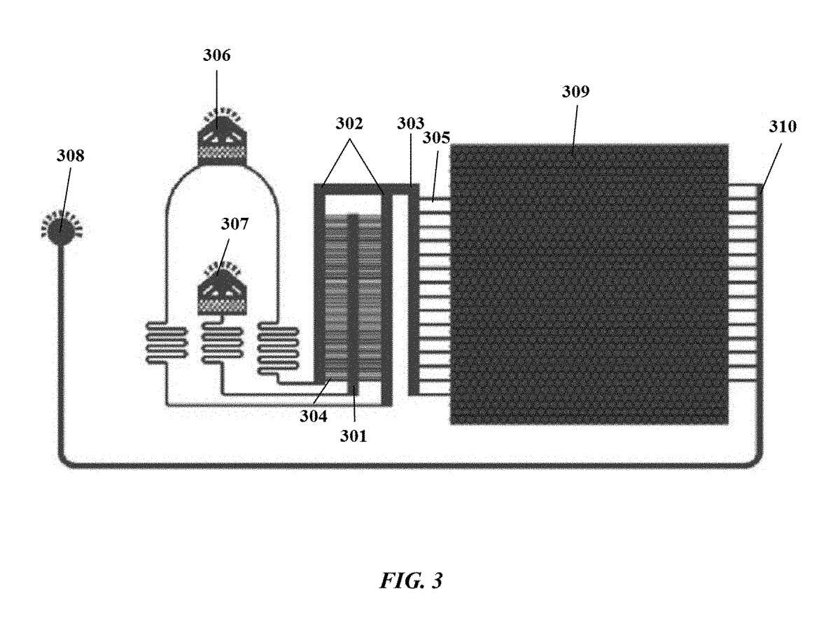

FIG. 3 provides a non-limiting example of the chip as described in the present disclosure. The chip comprises a first channel with a main channel 301 and a plurality of secondary channels 304. The main channel 301 of the first channel is in fluid communication with a reservoir 307. The reservoir 307 may be filled with an aqueous fluid comprising a nucleic acid sample to be analyzed, the aqueous fluid may further comprise reagents necessary for nucleic acid amplification. The chip comprises two second channels 302 in fluid communication with each other. The second channels 302 are also in fluid communication with a reservoir 306. The reservoir 306 may be filled with a non-aqueous fluid (e.g., a fluorinated oil). The chip further comprises a third channel including a main channel 303 and a plurality of secondary channels 305. The third channel is in fluid communication with the second channels 302 and a collection area 309. The collection area 309 is connected to a reservoir 308 through a fourth channel 310, the reservoir 308 may be used as a container to collect wastes and excess liquids. Inlets are provided at the site of the reservoirs 306 and 307, respectively, and an outlet is provided at the site of the reservoir 308. A pump or other suitable apparatus may be used to generate negative and/or positive pressure(s) at the inlets and/or outlet, such that a pressure difference (e.g., a pressure drop) is generated between the inlets and the outlet to drive the fluids to flow from the inlets through the channels to the outlet. The reservoirs 306, 307 and 308 may be bound to the chip via one or more connectors using various methods, such as ultrasonic bonding, laser bonding, etc. In some embodiments, V-shaped connectors are used to bind a reservoir to the chip to facilitate flow of fluids getting into and out of the chip. In an example, an aqueous fluid comprising a nucleic acid sample and reagents necessary for nucleic acid amplification (e.g., buffers, polymerases, primers, probes, dNTPs, etc.) may be filled into the reservoir 307 and then directed to the first channel 301, meanwhile, a non-aqueous fluid may be filled into the reservoir 306 and then directed to the second channels 302. In some embodiments, the reservoir 307 is provided with a filter (e.g., a filtration membrane) to remove undesired components from the sample. The aqueous fluid is driven to pass through the plurality of secondary channels 304 towards a plurality of intersections formed between the plurality of secondary channels 304 and the second channels 302. Upon contacting between the aqueous fluid and the non-aqueous fluid at the plurality of intersections, a plurality of droplets is formed with each droplet containing a portion of the aqueous fluid surrounded by the non-aqueous fluid (e.g., a water-in-oil droplet). The plurality of droplets is then directed through the third channel 303 and its plurality of secondary channels 305 to the collection area 309. In some embodiments, the collection area 309 comprises one or more collection area inlets at one or more junctions with the third channel 305. In some embodiments, the collection area 309 comprises one or more collection area outlets at one or more junctions with the channel 310. The collection area 309 has a height that is no more than the average diameter of the droplets so that a single layer of droplets is present in the collection area, in which case each droplet is in direct contact with the bottom of the collection area, which in turn assures rapid and accurate temperature control of the droplets. After sufficient droplets enter the collection area, the force driving the movement of the droplets (e.g., generated by pressure drop) is removed to keep the droplets substantially stationary in the collection area. Then, the nucleic acid sample or portion thereof in each of the plurality of droplets is subjected to a nucleic acid amplification reaction under conditions that are sufficient to yield an amplification product(s) of the nucleic acid sample or a portion thereof. For example, the droplets may be subjected to thermal cycling between a first temperature (e.g., from about 50° C. to about 60° C.) and a second temperature (from about 92° C. to about 95° C.) for about 40 cycles. Then, excitation energy (e.g., high-power light-emitting diode or laser) is directed to the droplets to generate optical signals simultaneously in the majority of the droplets (in some embodiments, in all the droplets). In some embodiments, the optical signals may be fluorescent signals. In some embodiments, electrochemical signals or electrostatic signals may be generated. A signal detector (e.g., a CCD camera) may be used to detect and record signals generated from the droplets (e.g., by taking one or more pictures). The pictures captured may then be integrated into a single image reflecting amplification results of the droplets comprised in the collection area, indicating presence or absence of a target nucleic acid in each droplet. After detection of the signals, the droplets may be driven to flow out of the collection area through the outlets into the channel 310, and then into the reservoir 308 to be discarded or for further treatment.

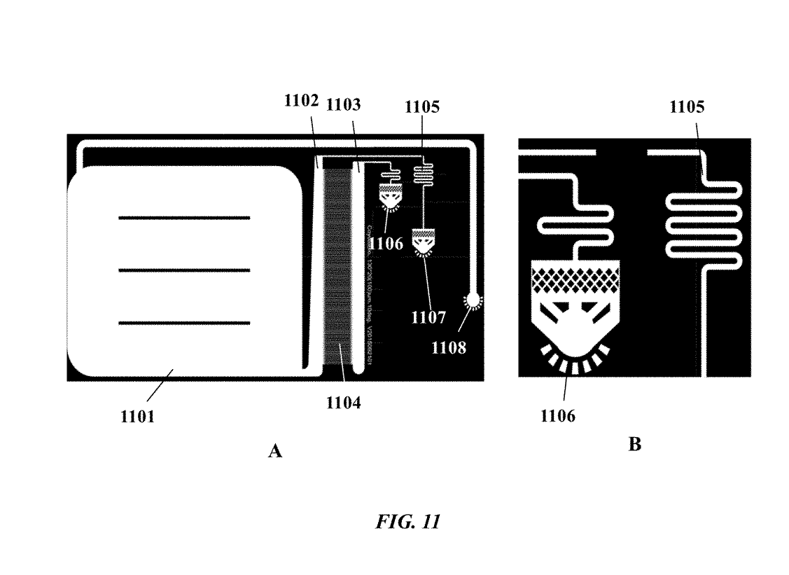

FIG. 11 provides another example of the chip as described in the present disclosure. The chip comprises a first channel with a main channel 1103 and a plurality of secondary channels 1104. The main channel 1103 of the first channel is in fluid communication with a reservoir 1106. The reservoir 1106 may be filled with an aqueous fluid comprising a nucleic acid sample to be analyzed, the aqueous fluid may further comprise reagents necessary for nucleic acid amplification. The chip comprises a second channels 1102 in fluid communication with the first channel. The second channel 1102 is also in fluid communication with a reservoir 1107. The reservoir 1107 may be filled with a non-aqueous fluid (e.g., a fluorinated oil). The second channel 1102 is in fluid communication with a collection area 1101. The collection area 1101 is connected to a reservoir 1108, the reservoir 1108 may be used as a container to collect wastes and excess liquids. Inlets are provided at the site of the reservoirs 1106 and 1107, respectively, and an outlet is provided at the site of the reservoir 1108. A pump or other suitable apparatus may be used to generate negative and/or positive pressure(s) at the inlets and/or outlet, such that a pressure difference (e.g., a pressure drop) is generated between the inlets and the outlet to drive the fluids to flow from the inlets through the channels to the outlet. The reservoirs 1106, 1107 and 1108 may be bound to the chip via one or more connectors using various methods, such as ultrasonic bonding, laser bonding, etc. In some embodiments, V-shaped connectors are used to bind a reservoir to the chip to facilitate flow of fluids getting into and out of the chip. Inlet of the second channel 1102 may comprise a hydrodynamic resistor 1105, which is also in fluid communication with the reservoir 1107, thereby flow of the non-aqueous fluid from the reservoir 1107 to the second channel 1102 may be adjusted through the hydrodynamic resistor 1105. In an example, an aqueous fluid comprising a nucleic acid sample and reagents necessary for nucleic acid amplification (e.g., buffers, polymerases, primers, probes, dNTPs, etc.) may be filled into the reservoir 1106 and then directed to the first channel 1103, meanwhile, a non-aqueous fluid may be filled into the reservoir 1107 and then directed to the second channel 1102. In some embodiments, the reservoir 1107 is provided with a filter (e.g., a filtration membrane) to remove undesired components from the sample. The aqueous fluid is driven to pass through the plurality of secondary channels 1104 towards a plurality of intersections formed between the plurality of secondary channels 1104 and the second channel 1102. Upon contacting between the aqueous fluid and the non-aqueous fluid at the plurality of intersections, a plurality of droplets is formed with each droplet containing a portion of the aqueous fluid surrounded by the non-aqueous fluid (e.g., a water-in-oil droplet). The plurality of droplets is then directed to the collection area 1101. The collection area 1101 has a height that is no more than the average diameter of the droplets so that no more than three layers (e.g., two layers or a single layer) of droplets are present in the collection area, in which case a majority of the droplets are in direct contact with the bottom of the collection area, which in turn assures rapid and accurate temperature control of the droplets. After sufficient droplets enter the collection area, the force driving the movement of the droplets (e.g., generated by pressure drop) is removed to keep the droplets substantially stationary in the collection area. Then, the nucleic acid sample or portion thereof in each of the plurality of droplets is subjected to a nucleic acid amplification reaction under conditions that are sufficient to yield an amplification product(s) of the nucleic acid sample or a portion thereof. For example, the droplets may be subjected to thermal cycling between a first temperature (e.g., from about 50° C. to about 60° C.) and a second temperature (from about 92° C. to about 95° C.) for about 40 cycles. Then, excitation energy (e.g., high-power light-emitting diode or laser) is directed to the droplets to generate optical signals simultaneously in the majority of the droplets (in some embodiments, in all the droplets). In some embodiments, the optical signals may be fluorescent signals. In some embodiments, electrochemical signals or electrostatic signals may be generated. A signal detector (e.g., a CCD camera) may be used to detect and record signals generated from the droplets (e.g., by taking one or more pictures). The pictures captured may then be integrated into a single image reflecting amplification results of the droplets comprised in the collection area, indicating presence or absence of a target nucleic acid in each droplet. After detection of the signals, the droplets may be driven to flow out of the collection area through the outlets into the reservoir 1108 to be discarded or for further treatment.

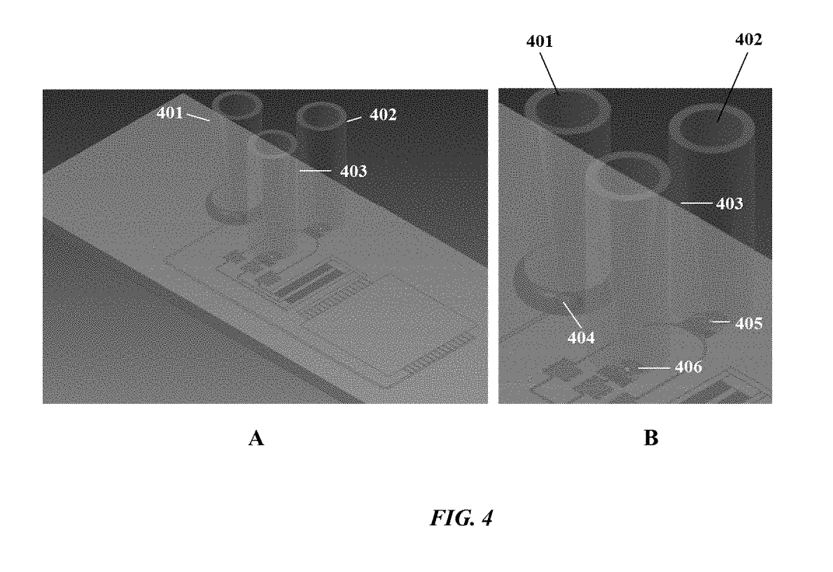

FIG. 4 (panel A) provides an example of reservoirs 401, 402 and 403 adjacent to a chip. FIG. 4 (panel B) shows an enlarged view of the reservoirs. Reservoir 401 may be used as a container to collect wastes and excess liquids. Reservoir 402 may be filled with a non-aqueous fluid (e.g., a fluorinated oil). Reservoir 403 may be filled with an aqueous fluid comprising a nucleic acid sample to be analyzed, the aqueous fluid may further comprise reagents necessary for nucleic acid amplification.

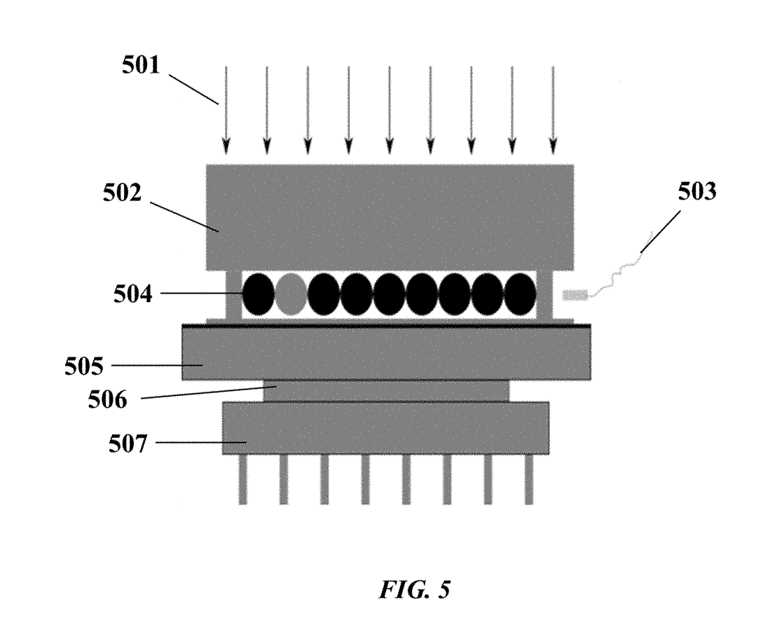

FIG. 5 shows an enlarged side view of a portion of a collection area according to an embodiment of the present disclosure. The collection area comprises a cover 502 and a substrate 505. The cover 502 and/or the substrate 505 may be transparent or non-transparent. The plurality of partitions 504 (e.g., the plurality of droplets) of the present disclosure may be comprised in a single layer between the cover 502 and the substrate 505. A heat-conducting/generating element 506 (e.g., a Peltier element) and a cooling element 507 may be attached to the bottom side of the substrate 505. The collection area may further comprise a temperature sensor 503 to monitor temperature changes therein. Excitation energy 501 may be directed to the collection area from above. In some embodiments, signals (e.g., optical signals, such as fluorescent signals) are detected by a detector located above the cover 502.

Various approaches may be used to generate partitions, such as droplets. In some examples, an aqueous fluid is brought in contact with a non-aqueous fluid, or vice versa, to generate partitions. The aqueous fluid can be directed along at least a first channel and the non-aqueous fluid can be directed along at least a second channel to an intersection of the first channel and second channel. At the intersection, contact between the aqueous fluid and the non-aqueous fluid can generate partitions, such as droplets.

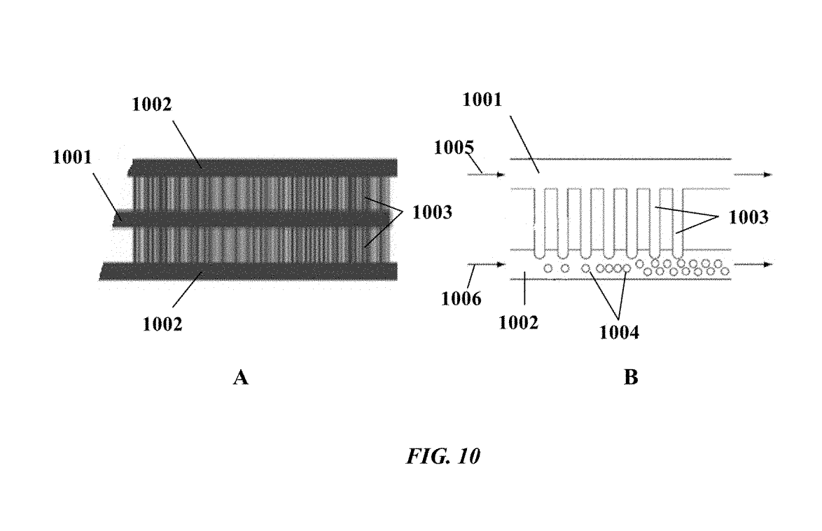

For example, the device shown in FIG. 10 may be used with a device, method or system of the present disclosure. FIG. 10 (panel A) provides an example of a device that may be used to generate partitions (e.g., droplets), and an enlarged view is shown in FIG. 10 (panel B). The device may comprise a first channel 1001, a second channel 1002 and a plurality of side channels 1003 each connecting the first channel with the second channel. Some or all of these channels may be microfluidic. The side channels 1003 may be substantially perpendicular to the second channel 1002. As an alternative, at least some or all of the side channels 1003 may be oriented at an angle that is at least about 10°, 20°, 25°, 30°, 40°, 45°, 50°, 60°, 70° 80°, or 85° with respect to the second channel 1002. A first fluid 1005 (e.g., an aqueous fluid) may enter through the first channel 1001 while a second fluid 1006 (e.g., a non-aqueous fluid) may enter through the second channel 1002. The first fluid 1005 may flow through the side channels 1003 to enter the second channel 1002. If the first fluid 1005 and the second fluid 1006 are at least substantially immiscible, the first fluid 1005 exiting the side channels 1003 may form individual partitions (e.g., droplets) 1004 within the second channel 1002. In addition, in some embodiments, the first fluid 1005 itself comprises emulsions, accordingly, double emulsions or emulsions of higher-order may be formed accordingly. In some cases, the partitions 1004 may have substantially the same size or characteristic dimension, for example, when the side channels 1003 have substantially the same cross-sectional area and/or length and/or other dimensions. In such a way, a plurality of substantially monodisperse partitions may be formed.

### System for Analyzing Nucleic Acid Samples

In another aspect, the present disclosure provides a system for analyzing a nucleic acid sample of a subject. The system may comprise a chip comprising a plurality of intersections of a first channel and a second channel, wherein during use, (1) the first channel directs an aqueous fluid comprising the nucleic acid sample and (2) the second channel directs a non-aqueous fluid towards the plurality of intersections, so as to form a plurality of partitions at the plurality of intersections upon contacting between the aqueous fluid and the non-aqueous fluid, wherein each of the plurality of partitions includes (i) the nucleic acid sample or portion thereof, and (ii) reagents necessary for nucleic acid amplification. In the second channel, the non-aqueous fluid may be substantially free of the sample and the reagents.

In one aspect, the present disclosure provides a system for analyzing a nucleic acid sample of a subject. The system may comprise a chip comprising a first channel and a second channel meeting at an intersection, wherein during use, (1) the first channel directs an aqueous fluid comprising the nucleic acid sample and (2) the second channel directs a non-aqueous fluid towards the intersection, so as to form a plurality of partitions at the intersection upon contacting between the aqueous fluid and the non-aqueous fluid, wherein each of the plurality of partitions includes (i) the nucleic acid sample or portion thereof, and (ii) reagents necessary for nucleic acid amplification. The system may further comprise one or more computer processors that are individually or collectively programmed to (i) subject the nucleic acid sample or portion thereof in each of the plurality of partitions to a nucleic acid amplification reaction under conditions that are sufficient to yield an amplification product(s) of the nucleic acid sample or portion thereof, and (ii) subsequent to (i), with the plurality of partitions disposed in a collection area that is substantially planar, simultaneously detect signals indicative of a presence or absence of the amplification product(s) in the plurality of partitions.

In another aspect, the present disclosure provides a system for analyzing a nucleic acid sample of a subject. The system may comprise a chip comprising a first channel and a second channel meeting at an intersection, wherein during use, (1) the first channel directs an aqueous fluid comprising the nucleic acid sample and (2) the second channel directs a non-aqueous fluid towards the intersection, so as to form a plurality of partitions at the intersection upon contacting between the aqueous fluid and the non-aqueous fluid, wherein each of the plurality of partitions includes (i) the nucleic acid sample or portion thereof, and (ii) reagents necessary for nucleic acid amplification. The system may further comprise one or more computer processors that are individually or collectively programmed to (i) subject the nucleic acid sample or portion thereof in each of the plurality of partitions to a nucleic acid amplification reaction under conditions that are sufficient to yield an amplification product(s) of the nucleic acid sample or portion thereof, and (ii) subsequent to (i), simultaneously detect signals indicative of a presence or absence of the amplification product(s) in the plurality of partitions while the plurality of partitions are immobilized by wells in a collection area, wherein each of the wells has a dimension (e.g., length, width, depth) that is less than an average diameter of a given partition of the plurality of partitions.

The aqueous fluid may comprise a nucleic acid sample and reagents necessary for nucleic acid amplification. The nucleic acid sample and reagents necessary for nucleic acid amplification are as described elsewhere in the present disclosure.

The non-aqueous fluid may comprise hydrophobic liquids. Non-limiting examples of the hydrophobic liquids include oils, such as hydrocarbons, silicon oils, fluorocarbon oils, organic solvents etc. In some embodiments, the oil is a fluorinated oil, such as HFE 7100, HFE 7500, FC-40, FC-43, FC-70, FC-3208, or a combination thereof. In some embodiments, the oil is a mineral oil, such as liquid paraffin, light mineral oil, white oil, refined mineral oil, cycloalkane oil, aromatic oil, or a combination thereof. The oil may also be any known oil that is useful for making droplets.

The non-aqueous fluid may comprise a surfactant. The surfactant may comprise a hydrophobic tail and a hydrophilic head group, a polymer-based tail and a hydrophilic head group, a polymer-based tail and a polymer-based head group, a fluorinated tail and a hydrophilic head group, or a fluorinated polymer-based tail and a hydrophilic polymer-based head group. In some embodiments, the surfactant is of a di-block copolymer or tri-block copolymer type. For example, the surfactant may be a block copolymer, such as a tri-block copolymer comprising two perfluoropolyether (PFPE) blocks and one poly(ethylene)glycol (PEG) block. In some embodiments, the surfactant is selected from the group consisting of perfluoropolyether-polyethylene glycol-perfluoropolyether (PFPE-PEG-PFPE), tri-block copolymer eicosylamine surfactant and dimorpholino phosphate surfactant (see, e.g., Baret, Kleinschmidt, et al., 2009). The length of PEG in a polymeric species, including a polymeric surfactant, may have any suitable length and may vary between different polymeric species that can be used. The surfactant may be present in the non-aqueous fluid with a concentration of 0.0001% to 5% (w/w), e.g., 0.001% to 4% (w/w), 0.01% to 3% (w/w), 0.1% to 2% (w/w), 0.1% to 1% (w/w). In some embodiments, the surfactant is present in the non-aqueous fluid with a concentration of at least about, at most about or about 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.2% (w/w), 1.4% (w/w), 1.6% (w/w), 1.8% (w/w), 2.0% (w/w), 2.5% (w/w), 3.0% (w/w), 3.5% (w/w), 4.0% (w/w), 4.5% (w/w), 5.0% (w/w), 7.0% (w/w), 10.0% (w/w), 15.0% (w/w), 20.0% (w/w) or more or less.

The first channel may include a main channel and a plurality of secondary channels that intersect the second channel at the plurality of intersections. The plurality of secondary channels may be oriented at an angle from about 45° and 100° with respect to the main channel and/or the second channel.

In some cases, the chip may comprise multiple sets of the first channel, second channel, and plurality of intersections.

The system may further comprise one or more computer processors that are individually or collectively programmed to (i) subject the nucleic acid sample or portion thereof in each of the plurality of partitions to a nucleic acid amplification reaction under conditions that are sufficient to yield an amplification product(s) of the nucleic acid sample or portion thereof, and (ii) with the plurality of partitions disposed in a collection area downstream of the plurality of intersections, simultaneously detect signals indicative of a presence or absence of the amplification product(s) in the plurality of partitions. In some cases, the one or more computer processors may be individually or collectively programmed to simultaneously detect signals indicative of a presence or absence of the amplification product(s) in all of the plurality of partitions.

The one or more computer processors may be individually or collectively programmed to direct the plurality of partitions to the collection area.

The system may further comprise a third channel for directing the plurality of partitions from the plurality of intersections to the collection area. The third channel may have a diameter that is greater than a cross-section of each of the plurality of partitions.

The one or more computer processors may be individually or collectively programmed to subject the nucleic acid sample or portion thereof in each of the plurality of partitions to the nucleic acid amplification reaction in the collection area.

The channels, the chips and the collection area are as described elsewhere in the present disclosure.

The collection area may include a plurality of zones, and the one or more computer processors may be individually or collectively programmed to simultaneously detect the signals from a given zone of the plurality of zones. For example, the collection area may be divided into 4 or 5 overlapping or non-overlapping zones, and a camera may take one picture at a time recording signals generated from one of the zones. Then, the pictures taken may be assembled together to demonstrate signals detected in the entire collection area.

The collection area may be included in the chip. For example, the collection area may be substantially planar. In some embodiments, the collection area is rotatable.

The collection area may be curvilinear, for example, the collection area may be circular or tilted.

In some embodiments, the collection area may be removable from the chip.

The collection area may be dimensioned to accommodate the plurality of partitions in a single layer. For example, the collection area may be dimensioned in a manner to avoid stacking of the plurality of partitions, as described elsewhere in the present disclosure.

The plurality of partitions may be droplets.

The collection area may comprise wells that are dimensioned to hold a single partition of the plurality of partitions. Each of the wells may have a dimension (e.g., length, width, depth) that is less than an average diameter of a given partition of the plurality of partitions.

The nucleic acid amplification reaction may be polymerase chain reaction (PCR). For example, the nucleic acid amplification reaction may be isothermal PCR. The nucleic acid amplification reaction is as described elsewhere in the present disclosure.

The reagents necessary for nucleic acid amplification may include a polymerizing enzyme and primers having sequence complementary with a target nucleic acid sequence. The target nucleic acid sequence may be associated with a disease.

The disease may be associated with a virus. For example, the virus may be selected from the group consisting of human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, and Varicella virus.

In some embodiments, the disease is cancer. Non-limiting examples of the cancers include, e.g., colorectal cancer, bladder cancer, ovarian cancer, testicular cancer, breast cancer, skin cancer, lung cancer, pancreatic cancer, stomach cancer, esophageal cancer, brain cancer, leukemia, liver cancer, endometrial cancer, prostate cancer, and head and neck cancer.

The target nucleic acid sequence may be associated with food safety. Food safety can be compromised by foodborne illness caused by pathogenic microbes. The pathogenic microbes may be bacteria, viruses, or parasites. Therefore, in some embodiments of the present disclosure, the target nucleic acid sequence is associated with a pathogenic bacterium, a pathogenic virus, or a pathogenic parasite that may compromise food safety.

In some embodiments, the food safety may be compromised by a pathogenic bacterium. Non-limiting examples of pathogenic bacteria include Campylobacter jejuni, Clostridium perfringens, Salmonella spp., Escherichia coli O157:H7 enterohemorrhagic (EHEC), Bacillus cereus, other virulent Escherichia coli such as enteroinvasive (EIEC), enteropathogenic (EPEC), enterotoxigenic (ETEC), enteroaggregative (EAEC or EAgEC), Listeria monocytogenes, Shigella spp., Staphylococcus aureus, Staphylococcal enteritis, Streptococcus, Vibrio cholerae, including O1 and non-O1, Vibrio parahaemolyticus, Vibrio vulnificus, Yersinia enterocolitica and Yersinia pseudotuberculosis, Brucella spp., Corynebacterium ulcerans, Coxiella burnetii or Q fever, Plesiomonas shigelloides, and the like. Sometimes the food safety is compromised by an enterotoxin secreted by a bacterium rather than the bacterium per se. Non-limiting examples of such enterotoxin-secreting bacteria include Staphylococcus aureus, Clostridium botulinum, Clostridium perfringens, Bacillus cereus, Pseudoalteromonas tetraodonis, Pseudomonas spp., Vibrio spp., and the like.

In some embodiments, the food safety may be compromised by a pathogenic virus. Non-limiting examples of pathogenic virus include Enterovirus, Hepatitis A, Hepatitis E, Norovirus, Rotavirus, and the like.

In some embodiments, the food safety may be compromised by a pathogenic parasite. Non-limiting examples of pathogenic parasite include Diphyllobothrium sp., Nanophyetus sp., Taenia saginata, Taenia solium, Fasciola hepatica, Anisakis sp., Ascaris lumbricoides, Eustrongylides sp., Trichinella spiralis, Trichuris trichiura, Acanthamoeba, Cryptosporidium parvum, Cyclospora cayetanensis, Entamoeba histolytica, Giardia lamblia, Sarcocystis hominis, Sarcocystis suihominis, Toxoplasma gondii, and the like.

The target nucleic acid sequence may be associated with prenatal testing. Prenatal testing may be conducted during gestation for detecting potential conditions, disorders or diseases associated with fetus. In some embodiments, the presence or the amount of the target nucleic acid sequence may be indicative of potential conditions, disorders or diseases in prenatal testing. Non-limiting conditions, disorders and diseases that may be detected in prenatal testing include spina bifida, cleft palate, Tay-Sachs disease, sickle cell anemia, thalassemia, cystic fibrosis, muscular dystrophy, fragile X syndrome, aneuploidy such as Down Syndrome (Trisomy 21), Edwards Syndrome (Trisomy 18), and Patau Syndrome (Trisomy 13), and the like.

The target nucleic acid sequence may be associated with genetic testing. Genetic testing may be conducted for various purposes, including, but not limited to detection of genetic disorders, forensic testing, molecular diagnosis, paternity/maternity testing, and the like. In some embodiments, the presence or the amount of the target nucleic acid sequence may be indicative of the result of a genetic testing.

The target nucleic acid sequence may be associated with cancer liquid biopsy. Cancer liquid biopsy is useful for detecting cancer by analyzing liquid samples from a subject (such as blood or bodily fluid) for indicators of cancers, such as circulating tumor cells or cell-free tumor nucleic acids. In some embodiments, the presence or the amount of the target nucleic acid sequence may be indicative of having cancer or being in the risk of having cancer in a cancer liquid biopsy. The cancer may be any cancer that can be diagnosed with a cancer liquid biopsy. Non-limiting examples of cancers that can be diagnosed with a cancer liquid biopsy include breast cancer, colon cancer, leukemia, lymphoma, stomach cancer, lung cancer, prostate cancer, and the like.

The partitions may include detectable moieties that permit detection of the signals. For example, the detectable moieties may be selected from the group consisting of TaqMan probes, TaqMan Tamara probes, TaqMan MGB probes, Lion probes, SYBR green, SYBR blue, DAPI, propidium iodine, Hoeste, SYBR gold, locked nucleic acid probes, and molecular beacons. Alternatively, the probe maybe any known probe that is useful in the context of the methods of the present disclosure. Other detectable moieties that may be used are as described elsewhere in the present disclosure.

The nucleic acid sample may be from a genome of the subject. The nucleic acid sample may be a cell free nucleic acid sample. For example, the nucleic acid sample may be cell free deoxyribonucleic acid.

The one or more computer processors may be individually or collectively programmed to subject the nucleic acid sample or portion thereof in each of the plurality of partitions to the nucleic acid amplification reaction on the chip. The amplification process is as described elsewhere in the present disclosure.

The one or more computer processors may be individually or collectively programmed to subject each of the plurality of partitions to thermal cycling to subject the nucleic acid sample or portion thereof in each of the plurality of partitions to the nucleic acid amplification reaction. The thermal cycling may comprise cycling a temperature of each of the plurality of partitions between a first temperature and a second temperature that is greater than the first temperature. In some cases, the thermal cycling may comprise cycling a temperature of each of the plurality of partitions between more than two different temperatures.

In some embodiments, a thermoelectric element (e.g., a Peltier element) is attached to one side (e.g., the lower side) of the collection area, the thermoelectric element may be in close contact with the collection area and may have an area that is large enough to cover at least the entire collection area.

In some embodiments, one side of the collection area (e.g., the bottom side) may be made of heat-absorbing material capable of converting light energy into heat. For example, the side of the collection area opposing to the side made of heat-absorbing material (e.g., the top side) may be made of material able to transmit light (e.g., transparent material). Accordingly, light (e.g., emitted from a infra-red (IR) lamp positioned above the collection area) may get through and be used to elevate the temperature in the collection area (e.g., to change the temperature in the partitions).

In some embodiments, one or more temperature sensors may be comprised in the collection area to monitor temperatures, e.g. in real-time. The temperature sensors may provide feedback information to a system controlling the energy source (e.g., the IR lamps). The control system receiving information from the temperature sensor may in turn be controlled by one or more computer processors that are individually or collectively programmed to adjust the energy source.

The one or more computer processors may be individually or collectively programmed to subject each of the plurality of partitions to thermal cycling using a source of thermal energy that is external to the chip. For example, the source of thermal energy may be an infrared energy source.

The one or more computer processors may be individually or collectively programmed to subject each of the plurality of partitions to thermal cycling using a source of thermal energy that is integrated with the chip. For example, the source of thermal energy may be a Peltier or resistive heating element. Alternatively, the source of thermal energy may be an induction heating element.

The one or more computer processors may be individually or collectively programmed to direct excitation energy to the plurality of partitions and detect the signals as emissions from the plurality of partitions. The signals may be detected using a detector that is integrated with the chip. In some cases, the signals may be detected using a detector that is external to the chip. For example, the detector may be a charge-coupled device camera.

The excitation energy may be provided by a source of excitation energy that is integrated with the chip. In some cases, the excitation energy may be provided by a source of excitation energy that is external to the chip. For example, the excitation energy may be provided by a light-emitting diode or a laser. The signals may be optical signals, fluorescent signals and/or electrostatic signals.

The one or more computer processors may be individually or collectively programmed to provide the nucleic acid sample in the first channel without sample purification and/or ribonucleic acid (RNA) extraction.

The one or more computer processors may be individually or collectively programmed to simultaneously detect the signals while the plurality of partitions is flowing at a flow rate less than about 5 ml/h through the collection area. In some embodiments, the plurality of partitions is flowing at a flow rate of less than about 4 ml/h, less than about 3 ml/h, less than about 2 ml/h, less than about 1 ml/h, less than about 0.5 ml/h, less than about 0.1 ml/h, or less through the collection area. In some cases, the one or more computer processors may be individually or collectively programmed to simultaneously detect the signals while the plurality of partitions is substantially stationary/not moving.

The one or more computer processors may be individually or collectively programmed to simultaneously detect the signals while the plurality of partitions is substantially stationary.

The one or more computer processors may be individually or collectively programmed to direct the plurality of partitions out of the collection area towards an outlet.

The outlet may be under negative pressure. In some cases, the negative pressure may be less than or about −5 bar, −4 bar, −3 bar, −2 bar, −1 bar, −0.9 bar, −0.8 bar, −0.7 bar, −0.6 bar, −0.5 bar, −0.4 bar, −0.3 bar, −0.2 bar, −0.1 bar, −0.05 bar, −0.04 bar, −0.03 bar, −0.02 bar, −0.01 bar, −0.005 bar, −0.001 bar, −0.0001 bar, or less. The first channel and/or second channel may be under positive pressure with respect to the outlet. In some cases, the one or more computer processors may be individually or collectively programmed to subject the aqueous fluid and non-aqueous fluid to flow using a pressure drop between the first channel and/or second channel, and the outlet, that is at least about is at least about 0.1 psi, at least about 0.5 psi, at least about 1 psi, at least about 5 psi, at least about 10 psi, at least about 15 psi, at least about 20 psi, at least about 30 psi, at least about 40 psi, at least about 50 psi, at least about 60 psi, at least about 70 psi, at least about 80 psi, at least about 90 psi, at least about 100 psi, at least about 150 psi, at least about 200 psi, at least about 250 psi, at least about 300 psi, at least about 350 psi, at least about 400 psi, at least about 450 psi, at least about 500 psi, at least about 750 psi or more. In some cases, the one or more computer processors may be individually or collectively programmed to subject the aqueous fluid and non-aqueous fluid to flow using a pressure drop between the first channel and/or second channel, and the outlet, that is at most about 750 psi, at least about 500 psi, at least about 450 psi, at least about 400 psi, at least about 350 psi, at least about 300 psi, at least about 250 psi, at least about 200 psi, at least about 150 psi, at least about 100 psi, at least about 90 psi, at least about 80 psi, at least about 70 psi, at least about 60 psi, at least about 50 psi, at least about 40 psi, at least about 30 psi, at least about 20 psi, at least about 15 psi, at least about 10 psi, at least about 5 psi, at least about 1 psi, at least about 0.5 psi, at least about 0.1 psi or less

The collection area may include an individually addressable location for each of the plurality of partitions.

The amplification product may be detected at a sensitivity of at least about 60%, at least 70%, at least 80%, at least 85%, at least 90%, at least 95%, at least 96%, at least 97%, at least 98%, at least 99% or higher. For example, the amplification product is detected at a sensitivity of at least 90%.

The amplification product may be detected at a specificity of at least about 60%, at least 70%, at least 80%, at least 85%, at least 90%, at least 95%, at least 96%, at least 97%, at least 98%, at least 99% or higher. For example, the amplification product is detected at a specificity of at least about 90%.

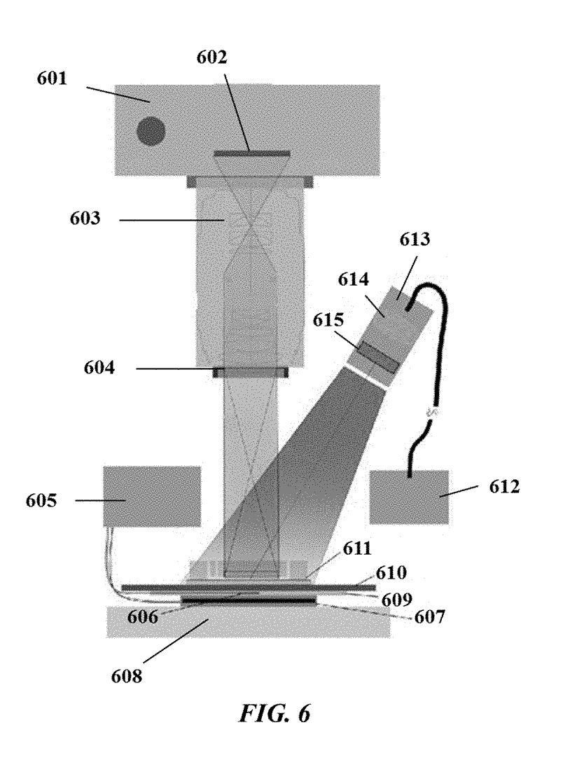

FIG. 6 shows an example of a system according to one embodiment of the present disclosure. A plurality of droplets are generated according to the present disclosure and directed to a collection area of a chip 611, on the bottom side, the chip is closely attached to a silicon wafer 610. The silicon wafer 610 may be attached to a copper plate 609 to form a thermoelectric element. Below the copper plate 609 is attached a thermo-electric cooler 607 for efficient cooling, which is also in contact with a heat block 608 to store or dissipate any additional heat. The chip 611 comprises a thermistor 606, which is connected with a thermo-cycle control module 605 to monitor and control temperatures of the chip and the droplets. Nucleic acid samples comprised in the droplets are amplified, as described in the present disclosure. Amplification may be on the chip 611 or off the chip 611. When the nucleic acid amplification reactions are completed, an excitation light is emitted by a light source 612. The light is directed to the droplets in the chip after being transmitted through an optic 613, one or more focusing lenses 614 and a filter 615. Signal moieties (e.g., those capable of generating fluorescent signals) are excited to generate detectable signals. The signals may then be transmitted through an emission filter 604, one or more lenses 603 to be detected by a sensor 602 in a camera 601. The camera 601 may record and/or analyze the signals captured and may transmit the detection information to one or more computer processors for further analysis.

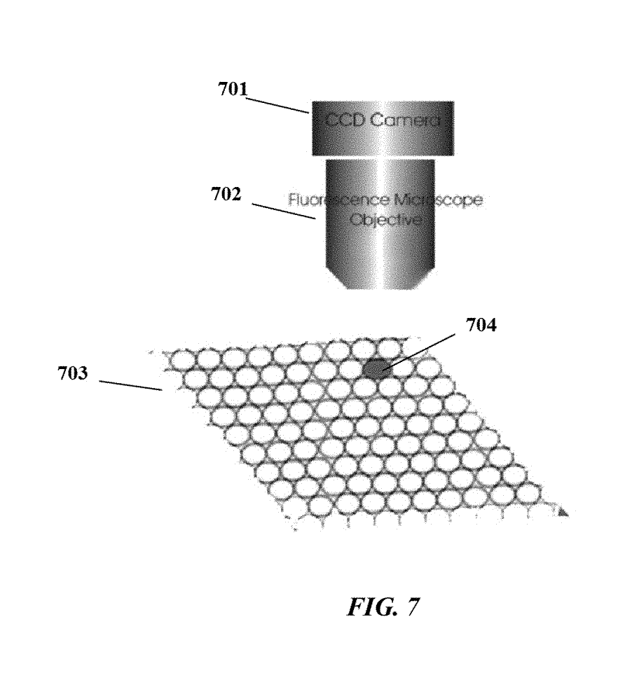

FIG. 7 shows an example of signal detection according the present disclosure. In this example, optical signals (e.g., fluorescent signals) generated from a droplet 704 comprised in a chip 703 may be detected and captured by a charge-coupled device (CCD) camera 701 with an objective 702. The objective 702 may be a microscope objective. The objective 702 may include one or more lens for observation of samples.

In some cases, signals from multiple droplets are detected simultaneously. This advantageously enables the rapid detection of an amplified product. As an alternative or in addition to, signals from groups of droplets may be detected simultaneously.

The system of the present disclosure may comprise an input module that receives a user request to analyze a nucleic acid sample obtained from a subject. Any suitable module capable of accepting such a user request may be used. The input module may comprise, for example, a device that comprises one or more processors. Non-limiting examples of devices that comprise processors (e.g., computer processors) include a desktop computer, a laptop computer, a tablet computer (e.g., Apple® iPad, Samsung® Galaxy Tab), a cell phone, a smart phone (e.g., Apple® iPhone, Android® enabled phone), a personal digital assistant (PDA), a video-game console, a television, a music playback device (e.g., Apple® iPod), a video playback device, a pager, and a calculator. Processors may be associated with one or more controllers, calculation units, and/or other units of a computer system, or implanted in firmware as desired. If implemented in software, the routines (or programs) may be stored in any computer readable memory such as in RAM, ROM, flash memory, a magnetic disk, a laser disk, or other storage medium. Likewise, this software may be delivered to a device via any delivery method including, for example, over a communication channel such as a telephone line, the internet, a local intranet, a wireless connection, etc., or via a transportable medium, such as a computer readable disk, flash drive, etc. The various steps may be implemented as various blocks, operations, tools, modules or techniques which, in turn, may be implemented in hardware, firmware, software, or any combination thereof. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

In some embodiments, the input module is configured to receive a user request to analyze a target nucleic acid. The input module may receive the user request directly (e.g. by way of an input device such as a keyboard, mouse, or touch screen operated by the user) or indirectly (e.g. through a wired or wireless connection, including over the internet). Via output electronics, the input module may provide the user's request to the one or more computer processors collectively or individually programmed to generate a plurality of partitions, subject the plurality of partitions to nucleic acid amplification reaction, detect any signals generated from an amplification product, and/or analyze data transmitted from a detector. In some embodiments, an input module may include a user interface (UI), such as a graphical user interface (GUI) that is configured to enable a user to provide a request to analyze the target nucleic acid. A GUI can include textual, graphical and/or audio components. A GUI can be provided on an electronic display, including the display of a device comprising a computer processor. Such a display may include a resistive or capacitive touch screen.

Non-limiting examples of users include the subject from which the nucleic acid sample was obtained, medical personnel, clinicians (e.g., doctors, nurses, and laboratory technicians), laboratory personnel (e.g., hospital laboratory technicians, research scientists, and pharmaceutical scientists), a clinical monitor for a clinical trial, or others in the health care industry.

In various aspects, the system comprises one or more computer processors individually or collectively programmed to perform analysis of a nucleic acid sample or a portion thereof, in response to a user request received by the input module. The one or more computer processors may be collectively or individually programmed to execute any of the methods described in the present disclosure.

In various aspects, a system of the present disclosure may comprise an output module operatively connected to the one or more computer processors. In some embodiments, the output module comprises a device with a processor as described above for the input module. The output module may include input devices as described herein and/or may comprise input electronics for communication with the one or more computer processors. In some embodiments, the output module is an electronic display, in some cases an electronic display comprising a UI. In some embodiments, the output module is a communication interface operatively coupled to a computer network such as, for example, the internet. In some embodiments, the output module transmits information to a recipient at a local or remote location using any suitable communication medium, including a computer network, a wireless network, a local intranet, or the internet. In some embodiments, the output module is capable of analyzing data received from the one or more computer processors or from a detector as described herein. In some cases, the output module includes a report generator capable of generating a report and transmitting the report to a recipient, wherein the report contains any information regarding the amount and/or presence of amplified product as described elsewhere herein. In some embodiments, the output module transmits information automatically in response to information received from the one or more computer processors or from a detector, such as in the form of raw data or data analysis performed by a software included in e.g., the detector. Alternatively, the output module may transmit information after receiving instructions from a user. Information transmitted by the output module may be viewed electronically or printed from a printer.

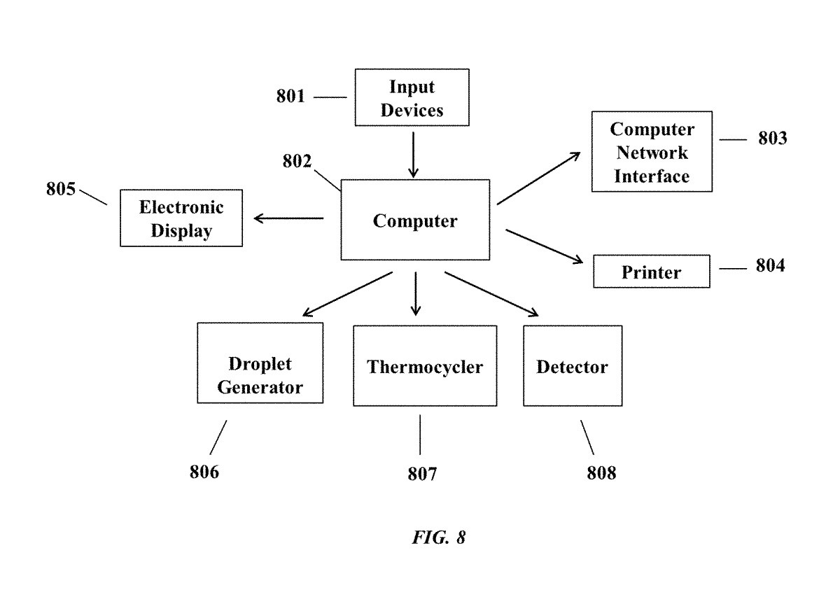

An example system for analyzing a nucleic acid sample according to the present disclosure is depicted in FIG. 8. A user may provide an aqueous fluid and a non-aqueous fluid in a droplet generator 806 comprised in a chip. The aqueous fluid may comprise a nucleic acid sample and reagents necessary for nucleic acid amplification. The system comprises input devices 801 (e.g., keyboard, mouse, etc.) that can receive the user's request to analyze the nucleic acid sample. The input devices 801 communicate the user's request to one or more processors in a computer 802. The one or more processors are individually or collectively programmed to generate droplets via the droplet generator 806, to subject the droplets generated to nucleic acid amplification via a thermocycler 807, and/or to detect any amplification signal generated via a detector 808. Information (e.g., raw data obtained by the detector) regarding the amplified product is transmitted from the detector 808 back to the computer 802. The computer 802 receives the information from the detector 808, performs any additional manipulations to the information, and then generates a report containing the processed information. Once the report is generated, the computer 802 then transmits the report to its end recipient over a computer network (e.g., an intranet, the internet) via computer network interface 803, in hard copy format via printer 804, or via the electronic display 805 operatively linked to computer 802.

### Computer Control Systems

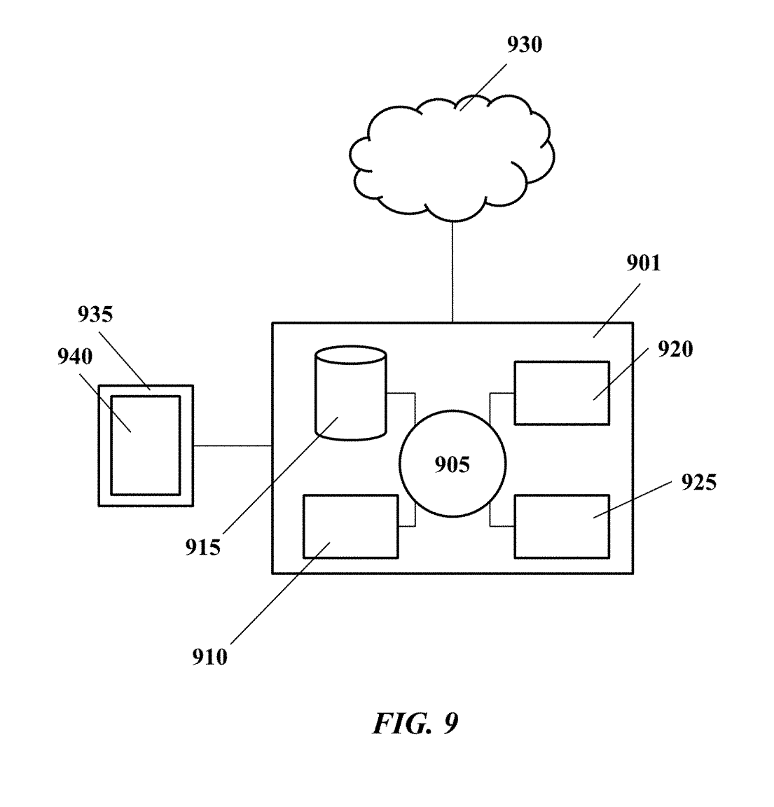

The present disclosure provides computer control systems that are programmed to implement methods of the disclosure. FIG. 9 shows a computer system 901 that is programmed or otherwise configured for nucleic acid sample processing and analysis, including nucleic acid amplification and detection. The computer system 901 can regulate various aspects of methods and systems of the present disclosure.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet. The network 930 in some cases is a telecommunication and/or data network. The network 930 can include one or more computer servers, which can enable distributed computing, such as cloud computing. The network 930, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processor adapted to execute the code, or can be compiled during runtime. The code can be supplied in a programming language that can be selected to enable the code to execute in a pre-compiled or as-compiled fashion.

In one aspect, the present disclosure provides a non-transitory computer-readable medium comprising machine executable code that, upon execution by one or more computer processors, implements a method for analyzing a nucleic acid sample of a subject. The method may comprise: (a) forming a plurality of partitions upon contact between an aqueous fluid comprising the nucleic acid sample and a non-aqueous fluid, wherein each of the plurality of partitions includes (i) the nucleic acid sample or portion thereof, and (ii) reagents necessary for nucleic acid amplification; (b) subjecting the nucleic acid sample or portion thereof in each of the plurality of partitions to a nucleic acid amplification reaction under conditions that are sufficient to yield an amplification product(s) of the nucleic acid sample or portion thereof, and (c) subsequent to (b), with the plurality of partitions disposed in a collection area that is substantially planar, simultaneously detecting signals indicative of a presence or absence of the amplification product(s) in the plurality of partitions.

In one aspect, the present disclosure provides a non-transitory computer-readable medium comprising machine executable code that, upon execution by one or more computer processors, implements a method for analyzing a nucleic acid sample of a subject. The method may comprise: (a) forming a plurality of partitions upon contact between an aqueous fluid comprising the nucleic acid sample and a non-aqueous fluid, wherein each of the plurality of partitions includes (i) the nucleic acid sample or portion thereof, and (ii) reagents necessary for nucleic acid amplification; (b) subjecting the nucleic acid sample or portion thereof in each of the plurality of partitions to a nucleic acid amplification reaction under conditions that are sufficient to yield an amplification product(s) of the nucleic acid sample or portion thereof; and (c) subsequent to (b), simultaneously detecting signals indicative of a presence or absence of the amplification product(s) in the plurality of partitions while the plurality of partitions are immobilized by wells in a collection area, wherein each of the wells has a dimension (e.g., length, width, depth) that is less than an average diameter of a given partition of the plurality of partitions.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing, for example, nucleic acid sequence information. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 905. The algorithm can, for example, regulate systems or implement methods provided herein.

Devices, methods and systems of the present disclosure may be combined with or modified by other devices, methods and systems, such as, for example, those described in WO/2014186440 (“APPARATUS AND METHOD FOR THE RAPID PRODUCTION OF DROPLETS”), which is entirely incorporated herein by reference.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-153. (canceled)

154. A method for analyzing a nucleic acid sample of a subject, comprising:
(a) directing (1) an aqueous fluid comprising said nucleic acid sample through a first channel and (2) a non-aqueous fluid through a second channel towards a plurality of intersections in a chip, so as to form a plurality of partitions at said plurality of intersections upon contacting between said aqueous fluid and said non-aqueous fluid, wherein each of said plurality of partitions includes (i) said nucleic acid sample or portion thereof, and (ii) reagents necessary for nucleic acid amplification;
(b) subjecting said nucleic acid sample or portion thereof in each of said plurality of partitions to a nucleic acid amplification reaction under conditions that are sufficient to yield an amplification product(s) of said nucleic acid sample or portion thereof; and
(c) with said plurality of partitions disposed in a collection area downstream of said plurality of intersections, simultaneously detecting signals indicative of a presence or absence of said amplification product(s) in said plurality of partitions.

155. The method of claim 154, further comprising directing said plurality of partitions to said collection area.

156. The method of claim 155, further comprising a third channel for directing said plurality of partitions from said plurality of intersections to said collection area.

157. The method of claim 156, wherein said third channel has a diameter that is greater than a cross-section of each of said plurality of partitions.

158. The method of claim 154, wherein (b) is performed in said collection area.

159. The method of claim 154, wherein said collection area is included in said chip, is substantially planar, is removable from said chip, or is dimensioned to accommodate said plurality of partitions in a single layer.

160. The method of claim 154, wherein said plurality of partitions is a plurality of droplets.

161. The method of claim 154, wherein (b) is performed on said chip.

162. The method of claim 154, wherein (b) comprises subjecting each of said plurality of partitions to thermal cycling.

163. The method of claim 162, wherein each of said plurality of partitions is subjected to thermal cycling using a source of thermal energy that is external to said chip.

164. The method of claim 162, wherein each of said plurality of partitions is subjected to thermal cycling using a source of thermal energy that is integrated with said chip.

165. The method of claim 154, wherein said collection area comprises wells that are dimensioned to hold a single partition of said plurality of partitions.

166. The method of claim 165, wherein each of said wells has a dimension that is less than an average diameter of a given partition of said plurality of partitions.

167. The method of claim 154, wherein said non-aqueous fluid comprises an oil or a surfactant.

168. The method of claim 154, wherein in said second channel, said non-aqueous fluid is substantially free of said sample and said reagents.

169. The method of claim 154, wherein said nucleic acid amplification reaction is polymerase chain reaction (PCR).

170. The method of claim 154, wherein said reagents include a polymerizing enzyme and primers having sequence complementary with a target nucleic acid sequence.

171. The method of claim 170, wherein said target nucleic acid sequence is associated with a disease, food safety, prenatal testing, genetic testing, or cancer liquid biopsy.

172. The method of claim 154, wherein said partitions include detectable moieties that permit detection of said signals.

173. The method of claim 154, wherein (c) comprises directing excitation energy to said plurality of partitions and detecting said signals as emissions from said plurality of partitions.

174. The method of claim 154, wherein said nucleic acid sample is from a genome of said subject.

175. The method of claim 154, wherein said nucleic acid sample is a cell free nucleic acid sample.

176. The method of claim 154, wherein in (c), said plurality of partitions is flowing at a flow rate less than about 5 milliliters per hour (ml/h) through said collection area.

177. The method of claim 176, wherein in (c), said plurality of partitions is substantially stationary.

178. The method of claim 177, wherein said first channel includes a main channel and a plurality of secondary channels that intersect said second channel at said plurality of intersections.

179. The method of claim 178, wherein said plurality of secondary channels are oriented at an angle from about 45° and 100° with respect to said main channel and/or said second channel.

180. The method of claim 154, wherein said chip comprises multiple sets of said first channel, second channel, and plurality of intersections.

181. The method of claim 154, further comprising, subsequent to (c), directing said plurality of partitions out of said collection area towards an outlet.

182. The method of claim 154, wherein at said collection area, each of said plurality of partitions is at an individually addressable location.

183. The method of claim 154, wherein said amplification product is detected at a sensitivity or a specificity of at least about 90%.

184. The method of claim 154, wherein (c) comprises simultaneously detecting signals indicative of a presence or absence of said amplification product(s) in all of said plurality of partitions.

185. A method for analyzing a nucleic acid sample of a subject, comprising:
(a) forming a plurality of partitions upon contact between an aqueous fluid comprising said nucleic acid sample and a non-aqueous fluid, wherein each of said plurality of partitions includes (i) said nucleic acid sample or portion thereof, and (ii) reagents necessary for nucleic acid amplification;
(b) subjecting said nucleic acid sample or portion thereof in each of said plurality of partitions to a nucleic acid amplification reaction under conditions that are sufficient to yield an amplification product(s) of said nucleic acid sample or portion thereof; and
(c) subsequent to (b), with said plurality of partitions disposed in a collection area that is substantially planar, simultaneously detecting signals indicative of a presence or absence of said amplification product(s) in said plurality of partitions.

186. A method for analyzing a nucleic acid sample of a subject, comprising:
(a) forming a plurality of partitions upon contact between an aqueous fluid comprising said nucleic acid sample and a non-aqueous fluid, wherein each of said plurality of partitions includes (i) said nucleic acid sample or portion thereof, and (ii) reagents necessary for nucleic acid amplification;
(b) subjecting said nucleic acid sample or portion thereof in each of said plurality of partitions to a nucleic acid amplification reaction under conditions that are sufficient to yield an amplification product(s) of said nucleic acid sample or portion thereof; and
(c) subsequent to (b), simultaneously detecting signals indicative of a presence or absence of said amplification product(s) in said plurality of partitions while said plurality of partitions are immobilized by wells in a collection area, wherein each of said wells has a dimension that is less than an average diameter of a given partition of said plurality of partitions.
